SEQUENCE LISTING (20 sequences)

SEQ ID NO: 1 - DNA, 720 bp, Aequorea victoria
  misc_feature (1)..(720): GFP
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 2 - protein, 239 aa, Aequorea victoria
  MISC_FEATURE (1)..(239): GFP
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 3 - DNA, 798 bp, Escherichia coli
  misc_feature (1)..(795): NPTII
atggggattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactccaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacacatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctga

SEQ ID NO: 4 - protein, 265 aa, Escherichia coli
  MISC_FEATURE (1)..(265): NPTII
MGIEQDGLHAGSPAAWVERLFGYDWAQQTIGCSDAAVFRLSAQGRPVLFVKTDLSGALNELQDEAARLSWLATTGVPCAAVLDVVTEAGRDWLLLGEVPGQDLLSSHLAPAEKVSIMADAMRRLHTLDPATCPFDHQAKHRIERARTRMEAGLVDQDDLDEEHQGLAPAELFARLKARMPDGEDLVVTHGDACLPNIMVENGRFSGFIDCGRLGVADRYQDIALATRDIAEELGGEWADRFLVLYGIAAPDSQRIAFYRLLDEFF

SEQ ID NO: 5 - DNA, 552 bp, Streptomyces viridochromogenes
  misc_feature (1)..(552): pat gene
atgagcccagaacgacgcccggtcgagatccgtcccgccaccgccgccgacatggcggcggtctgcgacatcgtcaatcactacatcgagacgagcacggtcaacttccgtacggagccgcagactccgcaggagtggatcgacgacctggagcgcctccaggaccgctacccctggctcgtcgccgaggtggagggcgtcgtcgccggcatcgcctacgccggcccctggaaggcccgcaacgcctacgactggaccgtcgagtcgacggtgtacgtctcccaccggcaccagcggctcggactgggctccaccctctacacccacctgctgaagtccatggaggcccagggcttcaagagcgtggtcgccgtcatcggactgcccaacgacccgagcgtgcgcctgcacgaggcgctcggatacaccgcgcgcgggacgctgcgggcagccggctacaagcacgggggctggcacgacgtggggttctggcagcgcgacttcgagctgccggccccgccccgccccgtccggcccgtcacacagatctga

SEQ ID NO: 6 - protein, 183 aa, Streptomyces viridochromogenes
  MISC_FEATURE (1)..(183): pat
MSPERRPVEIRPATAADMAAVCDIVNHYIETSTVNFRTEPQTPQEWIDDLERLQDRYPWLVAEVEGVVAGIAYAGPWKARNAYDWTVESTVYVSHRHQRLGLGSTLYTHLLKSMEAQGFKSVVAVIGLPNDPSVRLHEALGYTARGTLRAAGYKHGGWHDVGFWQRDFELPAPPRPVRPVTQI

SEQ ID NO: 7 - DNA, 1026 bp, Escherichia coli
  misc_feature (1)..(1026): hptII
atgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggagtttagcgagagcctgacctattgcatctcccgccgtgcacagggtgtcacgttgcaagacctgcctgaaaccgaactgcccgctgttctacaaccggtcgcggaggctatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccacgactccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaagaaatag

SEQ ID NO: 8 - protein, 341 aa, Escherichia coli
  MISC_FEATURE (1)..(341): hptII
MKKPELTATSVEKFLIEKFDSVSDLMQLSEGEESRAFSFDVGGRGYVLRVNSCADGFYKDRYVYRHFASAALPIPEVLDIGEFSESLTYCISRRAQGVTLQDLPETELPAVLQPVAEAMDAIAAADLSQTSGFGPFGPQGIGQYTTWRDFICAIADPHVYHWQTVMDDTVSASVAQALDELMLWAEDCPEVRHLVHADFGSNNVLTDNGRITAVIDWSEAMFGDSQYEVANIFFWRPWLACMEQQTRYFERRHPELAGSPRLRAYMLRIGLDQLYQSLVDGNFDDAAWAQGRCDAIVRSGAGTVGRTQIARRSAAVWTDGCVEVLADSGNRRPSTRPRAKK

SEQ ID NO: 9 - DNA, 10711 bp, artificial
  synthetic sequence
  misc_feature (1)..(10711): 131440 plant transformation vector
catgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccgcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtgacaggatatattgttgtgttaacaaattgacgcttagacaacttaataacacattgcggacgtttttaatgtactgaattaacgccgaattaattcgggggatctggattttagtactggattttggttttaggaattagaaattttattgatagaagtattttacaaatacaaatacatactaagggtttcttatatgctcaacacatgagcgaaaccctataggaaccctaattcccttatctgggaactactcacacattattatggagaaactcgagcttgtcgatcgactctagctagaggatcgatccgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgtgtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctggagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatccccatggtcgatcgacagatctgcgaaagctcgagagagatagatttgtagagagagactggtgatttcagcgtgtcctctccaaatgaaatgaacttccttatatagaggaaggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttggcaagctgctctagccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattcgagctctaactataacggtcctaaggtagcgagcgatcgcgcgtattggctagagcagcttgccaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacacgctgaaatcaccagtctctctctacaaatctatctctgtttaaacatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaacggaccggatctgtcgatcgacaagctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcccccgaattagcggccgcattaccctgttatccctactttcggattataacatcacatctatgtcgggtgcggagaaagaggtaatgaaatggcatgacgatcaaccataccagataactatgactctcttaaggtagccaaataatacccggtctgaacgaggtggcaaacagctattatgggtattatgggtggtacccggggatcctctagagtcgacctgcaggcatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgctagagcagcttgagcttggatcagattgtcgtttcccgccttcagtttaaactatcagtgtttgacaggatatattggcgggtaaacctaagagaaaagagcgtttattagaataacggatatttaaaagggcgtgaaaaggtttatccgttcgtccatttgtatgtg

SEQ ID NO: 10 - DNA, 13247 bp, artificial
  synthetic vector sequence
  misc_feature (1)..(13247): 130571 plant transformation vector
ttatccctactttcggattataacatcacatctatgtcgggtgcggagaaagaggtaatgaaatggcatgacgatcaaccataccagataactatgactctcttaaggtagccaaataatacccggtctgaacgaggtggcaaacagctattatgggtattatgggtggtacccggggatcctctagagtcgacctgcaggcatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgctagagcagcttgagcttggatcagattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggttcccctcgggagtgcttggcattccgtgcgataatgacttctgttcaaccacccaaacgtcggaaagcctgacgacggagcagcattccaaaaagatcccttggctcgtctgggtcggctagaaggtcgagtgggctgctgtggcttgatccctcaacgcggtcgcggacgtagcgcagcgccgaaaaatcctcgatcgcaaatccgacgctgtcgaaaagcgtgatctgcttgtcgctctttcggccgacgtcctggccagtcatcacgcgccaaagttccgtcacaggatgatctggcgcgagttgctggatctcgccttcaatccgggtctgtggcgggaactccacgaaaatatccgaacgcagcaagatcgtcgaccaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagggggggggggggggggacatgaggttgccccgtattcagtgtcgctgatttgtattgtctgaagttgtttttacgttaagttgatgcagatcaattaatacgatacctgcgtcataattgattatttgacgtggtttgatggcctccacgcacgttgtgatatgtagatgataatcattatcactttacgggtcctttccggtgatccgacaggttacggggcggcgacctcgcgggttttcgctatttatgaaaattttccggtttaaggcgtttccgttcttcttcgtcataacttaatgtttttatttaaaataccctctgaaaagaaaggaaacgacaggtgctgaaagcgagctttttggcctctgtcgtttcctttctctgtttttgtccgtggaatgaacaatggaacccccccccccccccccctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcagatcccccgatcaagtagatacactacatatatctacaatagacatcgagccggaaggtgatgtttactttcctgaaatccccagcaattttaggccagtttttacccaagacttcgcctctaacataaattatagttaccaaatctggcaaaagggttaacaagtggcagcaacggattcgcaaacctgtcacgccttttgtgccaaaagccgcgccaggtttgcgatccgctgtgccaggcgttaggcgtcatatgaagatttcggtgatccctgagcaggtggcggaaacattggatgctgagaaccatttcattgttcgtgaagtgttcgatgtgcacctatccgaccaaggctttgaactatctaccagaagtgtgagcccctaccggaaggattacatctcggatgatgactctgatgaagactctgcttgctatggcgcattcatcgaccaagagcttgtcgggaagattgaactcaactcaacatggaacgatctagcctctatcgaacacattgttgtgtcgcacacgcaccgaggcaaaggagtcgcgcacagtctcatcgaatttgcgaaaaagtgggcactaagcagacagctccttggcatacgattagagacacaaacgaacaatgtacctgcctgcaatttgtacgcaaaatgtggctttactctcggcggcattgacctgttcacgtataaaactagacctcaagtctcgaacgaaacagcgatgtactggtactggttctcgggagcacaggatgacgcctaacaattcattcaagccgacaccgcttcgcggcgcggcttaattcaggagttaaacatcatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctaggcaggcttatcttggacaagaagatcgcttggcctcgcgcgcagatcagttggaagaatttgttcactacgtgaaaggcgagatcaccaaggtagtcggcaaataatgtctaacaattcgttcaagccgacgccgcttcgcggcgcggcttaactcaagcgttagagagctggggaagactatgcgcgatctgttgaaggtggttctaagcctcgtacttgcgatggcatcggggcaggcacttgctgacctgccaattgttttagtggatgaagctcgtcttccctatgactactccccatccaactacgacatttctccaagcaactacgacaactccataagcaattacgacaatagtccatcaaattacgacaactctgagagcaactacgataatagttcatccaattacgacaatagtcgcaacggaaatcgtaggcttatatatagcgcaaatgggtctcgcactttcgccggctactacgtcattgccaacaatgggacaacgaacttcttttccacatctggcaaaaggatgttctacaccccaaaaggggggcgcggcgtctatggcggcaaagatgggagcttctgcggggcattggtcgtcataaatggccaattttcgcttgccctgacagataacggcctgaagatcatgtatctaagcaactagcctgctctctaataaaatgttaggcctcaacatctagtcgcaagctgaggggaaccactagtgtcatacgaacctccaagagacggttacacaaacgggtacattgttgatgtcatgtatgacaatcgcccaagtaagtatccagctgtgttcagaacgtacgtccgaattaattcatcggggtacggtcgacgatcgtcaacgttcacttctaaagaaatagcgccactcagcttcctcagcggctttatccagcgatttcctattatgtcggcatagttctcaagatcgacagcctgtcacggttaagcgagaaatgaataagaaggctgataattcggatctctgcgagggagatgatatttgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattcccgggacttccgattaagtagagcttcccctgccgaagcgcgtggagctacaacccaaacctgcagctgaattaacgccgaattaattcgggggatctggattttagtactggattttggttttaggaattagaaattttattgatagaagtattttacaaatacaaatacatactaagggtttcttatatgctcaacacatgagcgaaaccctataggaaccctaattcccttatctgggaactactcacacattattatggagaaactcgagtcagatctgtgtgacgggccggacggggcggggcggggccggcagctcgaagtcgcgctgccagaaccccacgtcgtgccagcccccgtgcttgtagccggctgcccgcagcgtcccgcgcgcggtgtatccgagcgcctcgtgcaggcgcacgctcgggtcgttgggcagtccgatgacggcgaccacgctcttgaagccctgggcctccatggacttcagcaggtgggtgtagagggtggagcccagtccgagccgctggtgccggtgggagacgtacaccgtcgactcgacggtccagtcgtaggcgttgcgggccttccaggggccggcgtaggcgatgccggcgacgacgccctccacctcggcgacgagccaggggtagcggtcctggaggcgctccaggtcgtcgatccactcctgcggagtctgcggctccgtacggaagttgaccgtgctcgtctcgatgtagtgattgacgatgtcgcagaccgccgccatgtcggcggcggtggcgggacggatctcgaccgggcgtcgttctgggctcataagcttatcgtctacctgcagaagtaacaccaaacaacagggtgagcatcgacaaaagaaacagtaccaagcaaataaatagcgtatgaaggcagggctaaaaaaatccacatatagctgctgcatatgccatcatccaagtatatcaagatcaaaataattataaaacatacttgtttattataatagataggtactcaaggttagagcatatgaatagatgctgcatatgccatcatgtatatgcatcagtaaaacccacatcaacatgtatacctatcctagatcgatatttccatccatcttaaactcgtaactatgaagatgtatgacacacacatacagttccaaaattaataaatacaccaggtagtttgaaacagtattctactccgatctagaacgaatgaacgaccgcccaaccacaccacatcatcacaaccaagcgaacaaaaagcatctctgtatatgcatcagtaaaacccgcatcaacatgtatacctatcctagatcgatatttccatccatcatcttcaattcgtaactatgaatatgtatggcacacacatacagatccaaaattaataaatccaccaggtagtttgaaacagaattctactccgatctagaacgaccgcccaaccagaccacatcatcacaaccaagacaaaaaaaagcatgaaaagatgacccgacaaacaagtgcacggcatatattgaaataaaggaaaagggcaaaccaaaccctatgcaacgaaacaaaaaaaatcatgaaatcgatcccgtctgcggaacggctagagccatcccaggattccccaaagagaaacactggcaagttagcaatcagaacgtgtctgacgtacaggtcgcatccgtgtacgaacgctagcagcacggatctaacacaaacacggatctaacacaaacatgaacagaagtagaactaccgggccctaaccatggaccggaacgccgatctagagaaggtagagagggggggggggggaggacgagcggcgtaccttgaagcggaggtgccgacgggtggatttgggggagatctggttgtgtgtgtgtgcgctccgaacaacacgaggttggggaaagagggtgtggagggggtgtctatttattacggcgggcgaggaagggaaagcgaaggagcggtgggaaaggaatcccccgtagctgccgtgccgtgagaggaggaggaggccgcctgccgtgccggctcacgtctgccgctccgccacgcaatttctggatgccgacagcggagcaagtccaacggtggagcggaactctcgagaggggtccagaggcagcgacagagatgccgtgccgtctgcttcgcttggcccgacgcgacgctgctggttcgctggttggtgtccgttagactcgtcgacggcgtttaacaggctggcattatctactcgaaacaagaaaaatgtttccttagtttttttaatttcttaaagggtatttgtttaatttttagtcactttattttattctattttatatctaaattattaaataaaaaaactaaaatagagttttagttttcttaatttagaggctaaaatagaataaaatagatgtactaaaaaaattagtctataaaaaccattaaccctaaaccctaaatggatgtactaataaaatggatgaagtattatataggtgaagctatttgcaaaaaaaaaggagaacacatgcacactaaaaagataaaactgtagagtcctgttgtcaaaatactcaattgtcctttagaccatgtctaactgttcatttatatgattctctaaaacactgatattattgtagtagtatagattatattattcgtagagtaaagtttaaatatatgtataaagatagataaactgcacttcaaacaagtgtgacaaaaaaaatatgtggtaattttttataacttagacatgcaatgctcattatctctagagaggggcacgaccgggtcacgctgcactgcagaagcttgctgagtggctccttcaacgttgcggttctgtcagttccaaacgtaaaacggcttgtcccgcgtcatcggcgggggtcataacgtgactcccttaattctccgctcatgatcagattgtcgtttcccgccttcagtttaaacgagctctaactataacggtcctaaggtagcgagcgatcgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatactactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcacggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtttaaacatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaacggaccggtcatgggtcgtttaagctgccgatgtgcctgcgtcgtctggtgccctctctccatatggaggttgtcaaagtatctgctgttcgtgtcatgagtcgtgtcagtgttggtttaataatggaccggttgtgttgtgtgtgcgtactacccagaactatgacaaatcatgaataagtttgatgtttgaaattaaagcctgtgctcattatgttctgtctttcagttgtctcctaatatttgcctgcaggtactggctatctaccgtttcttacttaggaggtgtttgaatgcactaaaactaatagttagtggctaaaattagttaaaacatccaaacaccatagctaatagttgaactattagctatttttggaaaattagttaatagtgaggtagttatttgttagctagctaattcaactaacaatttttagccaactaacaattagtttcagtgcattcaaacacccccttaatgttaacgtggttctatctaccgtctcctaatatatggttgattgttcggtttgttgctatgctattgggttctgattgctgctagttcttgctgaatccagaagttctcgtagtatagctcagattcatattatttatttgagtgatggcgcgcccgagtatcgaattcctgcaggcatgcaagcgatccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcggcggccgcattaccctg

SEQ ID NO: 11 - DNA, 13728 bp, artificial
  synthetic vector sequence
  misc_feature (1)..(13728): 130836 plant transformation vector
gagctctaactataacggtcctaaggtagcgagcgatcgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatactactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcacggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtttaaacatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaacggaccggtcatgggtcgtttaagctgccgatgtgcctgcgtcgtctggtgccctctctccatatggaggttgtcaaagtatctgctgttcgtgtcatgagtcgtgtcagtgttggtttaataatggaccggttgtgttgtgtgtgcgtactacccagaactatgacaaatcatgaataagtttgatgtttgaaattaaagcctgtgctcattatgttctgtctttcagttgtctcctaatatttgcctgcaggtactggctatctaccgtttcttacttaggaggtgtttgaatgcactaaaactaatagttagtggctaaaattagttaaaacatccaaacaccatagctaatagttgaactattagctatttttggaaaattagttaatagtgaggtagttatttgttagctagctaattcaactaacaatttttagccaactaacaattagtttcagtgcattcaaacacccccttaatgttaacgtggttctatctaccgtctcctaatatatggttgattgttcggtttgttgctatgctattgggttctgattgctgctagttcttgctgaatccagaagttctcgtagtatagctcagattcatattatttatttgagtgatggcgcgcccgagtatcgaattcctgcaggcatgcaagcgatccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcggcggccgcattaccctgttatccctactttcggattataacatcacatctatgtcgggtgcggagaaagaggtaatgaaatggcatgacgatcaaccataccagataactatgactctcttaaggtagccaaataatacccggtctgaacgaggtggcaaacagctattatgggtattatgggtggtacccggggatcctctagagtcgacctgcaggcatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgctagagcagcttgagcttggatcagattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggttcccctcgggagtgcttggcattccgtgcgataatgacttctgttcaaccacccaaacgtcggaaagcctgacgacggagcagcattccaaaaagatcccttggctcgtctgggtcggctagaaggtcgagtgggctgctgtggcttgatccctcaacgcggtcgcggacgtagcgcagcgccgaaaaatcctcgatcgcaaatccgacgctgtcgaaaagcgtgatctgcttgtcgctctttcggccgacgtcctggccagtcatcacgcgccaaagttccgtcacaggatgatctggcgcgagttgctggatctcgccttcaatccgggtctgtggcgggaactccacgaaaatatccgaacgcagcaagatcgtcgaccaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagggggggggggggggggacatgaggttgccccgtattcagtgtcgctgatttgtattgtctgaagttgtttttacgttaagttgatgcagatcaattaatacgatacctgcgtcataattgattatttgacgtggtttgatggcctccacgcacgttgtgatatgtagatgataatcattatcactttacgggtcctttccggtgatccgacaggttacggggcggcgacctcgcgggttttcgctatttatgaaaattttccggtttaaggcgtttccgttcttcttcgtcataacttaatgtttttatttaaaataccctctgaaaagaaaggaaacgacaggtgctgaaagcgagctttttggcctctgtcgtttcctttctctgtttttgtccgtggaatgaacaatggaacccccccccccccccccctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcagatcccccgatcaagtagatacactacatatatctacaatagacatcgagccggaaggtgatgtttactttcctgaaatccccagcaattttaggccagtttttacccaagacttcgcctctaacataaattatagttaccaaatctggcaaaagggttaacaagtggcagcaacggattcgcaaacctgtcacgccttttgtgccaaaagccgcgccaggtttgcgatccgctgtgccaggcgttaggcgtcatatgaagatttcggtgatccctgagcaggtggcggaaacattggatgctgagaaccatttcattgttcgtgaagtgttcgatgtgcacctatccgaccaaggctttgaactatctaccagaagtgtgagcccctaccggaaggattacatctcggatgatgactctgatgaagactctgcttgctatggcgcattcatcgaccaagagcttgtcgggaagattgaactcaactcaacatggaacgatctagcctctatcgaacacattgttgtgtcgcacacgcaccgaggcaaaggagtcgcgcacagtctcatcgaatttgcgaaaaagtgggcactaagcagacagctccttggcatacgattagagacacaaacgaacaatgtacctgcctgcaatttgtacgcaaaatgtggctttactctcggcggcattgacctgttcacgtataaaactagacctcaagtctcgaacgaaacagcgatgtactggtactggttctcgggagcacaggatgacgcctaacaattcattcaagccgacaccgcttcgcggcgcggcttaattcaggagttaaacatcatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctaggcaggcttatcttggacaagaagatcgcttggcctcgcgcgcagatcagttggaagaatttgttcactacgtgaaaggcgagatcaccaaggtagtcggcaaataatgtctaacaattcgttcaagccgacgccgcttcgcggcgcggcttaactcaagcgttagagagctggggaagactatgcgcgatctgttgaaggtggttctaagcctcgtacttgcgatggcatcggggcaggcacttgctgacctgccaattgttttagtggatgaagctcgtcttccctatgactactccccatccaactacgacatttctccaagcaactacgacaactccataagcaattacgacaatagtccatcaaattacgacaactctgagagcaactacgataatagttcatccaattacgacaatagtcgcaacggaaatcgtaggcttatatatagcgcaaatgggtctcgcactttcgccggctactacgtcattgccaacaatgggacaacgaacttcttttccacatctggcaaaaggatgttctacaccccaaaaggggggcgcggcgtctatggcggcaaagatgggagcttctgcggggcattggtcgtcataaatggccaattttcgcttgccctgacagataacggcctgaagatcatgtatctaagcaactagcctgctctctaataaaatgttaggcctcaacatctagtcgcaagctgaggggaaccactagtgtcatacgaacctccaagagacggttacacaaacgggtacattgttgatgtcatgtatgacaatcgcccaagtaagtatccagctgtgttcagaacgtacgtccgaattaattcatcggggtacggtcgacgatcgtcaacgttcacttctaaagaaatagcgccactcagcttcctcagcggctttatccagcgatttcctattatgtcggcatagttctcaagatcgacagcctgtcacggttaagcgagaaatgaataagaaggctgataattcggatctctgcgagggagatgatatttgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattcccgggacttccgattaagtagagcttcccctgccgaagcgcgtggagctacaacccaaacctgcagctgaattaacgccgaattaattcgggggatctggattttagtactggattttggttttaggaattagaaattttattgatagaagtattttacaaatacaaatacatactaagggtttcttatatgctcaacacatgagcgaaaccctataggaaccctaattcccttatctgggaactactcacacattattatggagaaactcgagtcagatctgtctatttctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagtcgtggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatagcctccgcgaccggttgtagaacagcgggcagttcggtttcaggcaggtcttgcaacgtgacaccctgtgcacggcgggagatgcaataggtcaggctctcgctaaactccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggctttttcatatctcattctacctgcagaagtaacaccaaacaacagggtgagcatcgacaaaagaaacagtaccaagcaaataaatagcgtatgaaggcagggctaaaaaaatccacatatagctgctgcatatgccatcatccaagtatatcaagatcaaaataattataaaacatacttgtttattataatagataggtactcaaggttagagcatatgaatagatgctgcatatgccatcatgtatatgcatcagtaaaacccacatcaacatgtatacctatcctagatcgatatttccatccatcttaaactcgtaactatgaagatgtatgacacacacatacagttccaaaattaataaatacaccaggtagtttgaaacagtattctactccgatctagaacgaatgaacgaccgcccaaccacaccacatcatcacaaccaagcgaacaaaaagcatctctgtatatgcatcagtaaaacccgcatcaacatgtatacctatcctagatcgatatttccatccatcatcttcaattcgtaactatgaatatgtatggcacacacatacagatccaaaattaataaatccaccaggtagtttgaaacagaattctactccgatctagaacgaccgcccaaccagaccacatcatcacaaccaagacaaaaaaaagcatgaaaagatgacccgacaaacaagtgcacggcatatattgaaataaaggaaaagggcaaaccaaaccctatgcaacgaaacaaaaaaaatcatgaaatcgatcccgtctgcggaacggctagagccatcccaggattccccaaagagaaacactggcaagttagcaatcagaacgtgtctgacgtacaggtcgcatccgtgtacgaacgctagcagcacggatctaacacaaacacggatctaacacaaacatgaacagaagtagaactaccgggccctaaccatggaccggaacgccgatctagagaaggtagagagggggggggggggaggacgagcggcgtaccttgaagcggaggtgccgacgggtggatttgggggagatctggttgtgtgtgtgtgcgctccgaacaacacgaggttggggaaagagggtgtggagggggtgtctatttattacggcgggcgaggaagggaaagcgaaggagcggtgggaaaggaatcccccgtagctgccgtgccgtgagaggaggaggaggccgcctgccgtgccggctcacgtctgccgctccgccacgcaatttctggatgccgacagcggagcaagtccaacggtggagcggaactctcgagaggggtccagaggcagcgacagagatgccgtgccgtctgcttcgcttggcccgacgcgacgctgctggttcgctggttggtgtccgttagactcgtcgacggcgtttaacaggctggcattatctactcgaaacaagaaaaatgtttccttagtttttttaatttcttaaagggtatttgtttaatttttagtcactttattttattctattttatatctaaattattaaataaaaaaactaaaatagagttttagttttcttaatttagaggctaaaatagaataaaatagatgtactaaaaaaattagtctataaaaaccattaaccctaaaccctaaatggatgtactaataaaatggatgaagtattatataggtgaagctatttgcaaaaaaaaaggagaacacatgcacactaaaaagataaaactgtagagtcctgttgtcaaaatactcaattgtcctttagaccatgtctaactgttcatttatatgattctctaaaacactgatattattgtagtagtatagattatattattcgtagagtaaagtttaaatatatgtataaagatagataaactgcacttcaaacaagtgtgacaaaaaaaatatgtggtaattttttataacttagacatgcaatgctcattatctctagagaggggcacgaccgggtcacgctgcactgcagaagcttgctgagtggctccttcaacgttgcggttctgtcagttccaaacgtaaaacggcttgtcccgcgtcatcggcgggggtcataacgtgactcccttaattctccgctcatgatcagattgtcgtttcccgccttcagtttaaac

SEQ ID NO: 12 - DNA, 12103 bp, Artificial
  synthetic vector sequence
  misc_feature (1)..(12103): 133337 vector
catgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccgcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtgacaggatatattgttgtgttaacaaattgacgcttagacaacttaataacacattgcggacgtttttaatgtagatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatctcgactctagctagaggatcgatccgaaccccagagtcccgctcatcaggcagccttcgtatcggagagttcgatcttcgcgcccagcccggccatcaggtccatgaactccgggaagctcgtggcgatcatcgtggcatcgtccaccgtgacagggttttccgacacgaggcccatgacgaggaagctcatggcgatgcggtgatcgagatgggtggcgacggcggcgcccgaggcgttgccgagccccttgccgtcagggcggccgcgcacgacgagcgacgtctcgccctcatcgcaatccacgccattgagcttgaggccattggcgacggccgagaggcggtcgctttccttgacgcggagttcttccagaccgttcatcacggtcgccccttccgcgaaggcggcggcgacagcgagaatcggatattcgtcgatcatcgaaggcgcgcggtcttccggcaccgtgacgcccttcagcgtggaggagcgaacgcgcaggtccgccacgtcttcgccgccggcaaggcgcgggttgatgacttcgatgtcggcgcccatttcctgcagcgtcaggatgaggccggtgcgggtggggttcatcagcacgttgaggatggtgacgtcggagcccggaacaagcagggccgcaaccagcgggaaggccgtcgaggacgggtcgcccggcacgtcgatgacttggccggtgagcttgccgcggccttccaggcggatggtgcgcacgccgtccgcatccgtctcgacggtaaggttggcgccaaagccctgcagcatcttttccgtatgatcgcgcgtcatgatcggctcgatgaccgtcgtgatgccgggcgtgttgaggccggcgagcagcacggcggacttcacctgtgcggaggccatcggcacgcggtaggtgatcggcgtcggcgtcttcggcccgcgcaaggtaacgggaagacggtcaccgtcttccgatttcacctgcacgcccatttcgcgcagcgggttcaacacgcggcccatcgggcgctttgtgagcgaggcgtcgccgatgaaggtgctgtcgaaatcgtagaccccgacgaggcccatggtcaggcggcagcccgtggcggcattgccgaaatcgagcggcgcctcaggcgccaggaggccgccattgccgacgccatcgatgatccaggtgtcgccttccttacggatcctggcgcccatggcctgcatggccttgcccgtattgatgacgtcctcgccttccagaaggccggtgatgcgcgtttcaccgctcgcgagaccgccgaacatgaaggaccggtgggagatcgacttgtcgccgggaatgcggacggttccggaaaggccagaggatttgcgggcggttgcgggccggctgcttgcaccgtgaagcatgcacgccgtggaaacagaagacatgaccttaagaggacgaagctcagagccaattaacgtcatcccactcttcttcaatccccacgacgacgaaatcggataagctcgtggatgctgctgcgtcttcagagaaaccgataagggagatttgcgttgactggatttcgagagattggagataagagatgggttctgcacaccattgcagattctgctaacttgcgccatggtcgatcgacagatctgcgaaagctcgagctgttaatcagaaaaactcagattaatcgacaaattcgatcgcacaaactagaaactaacaccagatctagatagaaatcacaaatcgaagagtaattattcgacaaaactcaaattatttgaacaaatcggatgatatctatgaaaccctaatcgagaattaagatgatatctaacgatcaaacccagaaaatcgtcttcgatctaagattaacagaatctaaaccaaagaacatatacgaaattgggatcgaacgaaaacaaaatcgaagattttgagagaataaggaacacagaaatttaccttgatcacggtagagagaattgagagaaagtttttaagattttgagaaattgaaatctgaattgtgaagaagaagagctctttgggtattgttttatagaagaagaagaagaaaagacgaggacgactaggtcacgagaaagctaaggcggtgaagcaatagctaataataaaatgacacgtgtattgagcgttgtttacacgcaaagttgtttttggctaattgccttatttttaggttgaggaaaagtatttgtgctttgagttgataaacacgactcgtgtgtgccggctgcaaccactttgacgccgtttattactgactcgtcgacaaccacaatttctaacggtcgtcataagatccagccgttgagatttaacgatcgttacgatttatatttttttagcattatcgttttattttttaaatatacggtggagctgaaaattggcaataattgaaccgtgggtcccactgcattgaagcgtatttcgtattttctagaattcttcgtgctttatttcttttcctttttgtttttttttgccatttatctaatgcaagtgggcttataaaatcagtgaatttcttggaaaagtaacttctttatcgtataacatattgtgaaattatccatttcttttaattttttagtgttattggatatttttgtatgattattgatttgcataggataatgacttttgtatcaagttggtgaacaagtctcgttaaaaaaggcaagtggtttggtgactcgatttattcttgttatttaattcatatatcaatggatcttatttggggcctggtccatatttaacactcgtgttcagtccaatgaccaataatattttttcattaataacaatgtaacaagaatgatacacaaaacattctttgaataagttcgctatgaagaagggaacttatccggtcctagatcatcagttcatacaaacctccatagagttcaacatcttaaacaagaatatcctgattgctccaccatgttggcaagctgctctagccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattcgagctctaactataacggtcctaaggtagcgagcgatcgcgcgtattggctagagcagcttgccaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacacgctgaaatcaccagtctctctctacaaatctatctctgtttaaacatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtctgagaaggatgagctctgacggaccggatctgtcgatcgacaagctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcccccgaattagcggccgcattaccctgttatccctactttcggattataacatcacatctatgtcgggtgcggagaaagaggtaatgaaatggcatgacgatcaaccataccagataactatgactctcttaaggtagccaaataatacccggtctgaacgaggtggcaaacagctattatgggtattatgggtggtacccggggatcctctagagtcgacctgcaggcatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgctagagcagcttgagcttggatcagattgtcgtttcccgccttcagtttaaactatcagtgtttgacaggatatattggcgggtaaacctaagagaaaagagcgtttattagaataacggatatttaaaagggcgtgaaaaggtttatccgttcgtccatttgtatgtg

SEQ ID NO: 13 - DNA, 1596 bp, Agrobacterium sp.
  misc_feature (1)..(1596): cp4 gene
atggcgcaagttagcagaatctgcaatggtgtgcagaacccatctcttatctccaatctctcgaaatccagtcaacgcaaatctcccttatcggtttctctgaagacgcagcagcatccacgagcttatccgatttcgtcgtcgtggggattgaagaagagtgggatgacgttaattggctctgagcttcgtcctcttaaggtcatgtcttctgtttccacggcgtgcatgcttcacggtgcaagcagccggcccgcaaccgcccgcaaatcctctggcctttccggaaccgtccgcattcccggcgacaagtcgatctcccaccggtccttcatgttcggcggtctcgcgagcggtgaaacgcgcatcaccggccttctggaaggcgaggacgtcatcaatacgggcaaggccatgcaggccatgggcgccaggatccgtaaggaaggcgacacctggatcatcgatggcgtcggcaatggcggcctcctggcgcctgaggcgccgctcgatttcggcaatgccgccacgggctgccgcctgaccatgggcctcgtcggggtctacgatttcgacagcaccttcatcggcgacgcctcgctcacaaagcgcccgatgggccgcgtgttgaacccgctgcgcgaaatgggcgtgcaggtgaaatcggaagacggtgaccgtcttcccgttaccttgcgcgggccgaagacgccgacgccgatcacctaccgcgtgccgatggcctccgcacaggtgaagtccgccgtgctgctcgccggcctcaacacgcccggcatcacgacggtcatcgagccgatcatgacgcgcgatcatacggaaaagatgctgcagggctttggcgccaaccttaccgtcgagacggatgcggacggcgtgcgcaccatccgcctggaaggccgcggcaagctcaccggccaagtcatcgacgtgccgggcgacccgtcctcgacggccttcccgctggttgcggccctgcttgttccgggctccgacgtcaccatcctcaacgtgctgatgaaccccacccgcaccggcctcatcctgacgctgcaggaaatgggcgccgacatcgaagtcatcaacccgcgccttgccggcggcgaagacgtggcggacctgcgcgttcgctcctccacgctgaagggcgtcacggtgccggaagaccgcgcgccttcgatgatcgacgaatatccgattctcgctgtcgccgccgccttcgcggaaggggcgaccgtgatgaacggtctggaagaactccgcgtcaaggaaagcgaccgcctctcggccgtcgccaatggcctcaagctcaatggcgtggattgcgatgagggcgagacgtcgctcgtcgtgcgcggccgccctgacggcaaggggctcggcaacgcctcgggcgccgccgtcgccacccatctcgatcaccgcatcgccatgagcttcctcgtcatgggcctcgtgtcggaaaaccctgtcacggtggacgatgccacgatgatcgccacgagcttcccggagttcatggacctgatggccgggctgggcgcgaagatcgaactctccgatacgaaggctgcctga

SEQ ID NO: 14 - protein, 531 aa, Agrobacterium sp.
  MISC_FEATURE (1)..(531): CP4 protein
MAQVSRICNGVQNPSLISNLSKSSQRKSPLSVSLKTQQHPRAYPISSSWGLKKSGMTLIGSELRPLKVMSSVSTACMLHGASSRPATARKSSGLSGTVRIPGDKSISHRSFMFGGLASGETRITGLLEGEDVINTGKAMQAMGARIRKEGDTWIIDGVGNGGLLAPEAPLDFGNAATGCRLTMGLVGVYDFDSTFIGDASLTKRPMGRVLNPLREMGVQVKSEDGDRLPVTLRGPKTPTPITYRVPMASAQVKSAVLLAGLNTPGITTVIEPIMTRDHTEKMLQGFGANLTVETDADGVRTIRLEGRGKLTGQVIDVPGDPSSTAFPLVAALLVPGSDVTILNVLMNPTRTGLILTLQEMGADIEVINPRLAGGEDVADLRVRSSTLKGVTVPEDRAPSMIDEYPILAVAAAFAEGATVMNGLEELRVKESDRLSAVANGLKLNGVDCDEGETSLVVRGRPDGKGLGNASGAAVATHLDHRIAMSFLVMGLVSENPVTVDDATMIATSFPEFMDLMAGLGAKIELSDTKAA

SEQ ID NO: 15 - DNA, 738 bp, Aequorea victoria
  misc_feature (1)..(738): GFP-SEKDEL
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtctgagaaggatgagctctga

SEQ ID NO: 16 - protein, 245 aa, Aequorea victoria
  MISC_FEATURE (1)..(245): GFP-SEKDEL
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKSEKDEL

SEQ ID NO: 17 - DNA, 11302 bp, Artificial
  synthetic vector sequence
  misc_feature (1)..(11302): 133336 vector
catgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccgcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtgacaggatatattgttgtgttaacaaattgacgcttagacaacttaataacacattgcggacgtttttaatgtagatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatctcgactctagctagaggatcgatccgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgtgtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctggagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatccccatggtcgatcgacagatctgcgaaagctcgagctgttaatcagaaaaactcagattaatcgacaaattcgatcgcacaaactagaaactaacaccagatctagatagaaatcacaaatcgaagagtaattattcgacaaaactcaaattatttgaacaaatcggatgatatctatgaaaccctaatcgagaattaagatgatatctaacgatcaaacccagaaaatcgtcttcgatctaagattaacagaatctaaaccaaagaacatatacgaaattgggatcgaacgaaaacaaaatcgaagattttgagagaataaggaacacagaaatttaccttgatcacggtagagagaattgagagaaagtttttaagattttgagaaattgaaatctgaattgtgaagaagaagagctctttgggtattgttttatagaagaagaagaagaaaagacgaggacgactaggtcacgagaaagctaaggcggtgaagcaatagctaataataaaatgacacgtgtattgagcgttgtttacacgcaaagttgtttttggctaattgccttatttttaggttgaggaaaagtatttgtgctttgagttgataaacacgactcgtgtgtgccggctgcaaccactttgacgccgtttattactgactcgtcgacaaccacaatttctaacggtcgtcataagatccagccgttgagatttaacgatcgttacgatttatatttttttagcattatcgttttattttttaaatatacggtggagctgaaaattggcaataattgaaccgtgggtcccactgcattgaagcgtatttcgtattttctagaattcttcgtgctttatttcttttcctttttgtttttttttgccatttatctaatgcaagtgggcttataaaatcagtgaatttcttggaaaagtaacttctttatcgtataacatattgtgaaattatccatttcttttaattttttagtgttattggatatttttgtatgattattgatttgcataggataatgacttttgtatcaagttggtgaacaagtctcgttaaaaaaggcaagtggtttggtgactcgatttattcttgttatttaattcatatatcaatggatcttatttggggcctggtccatatttaacactcgtgttcagtccaatgaccaataatattttttcattaataacaatgtaacaagaatgatacacaaaacattctttgaataagttcgctatgaagaagggaacttatccggtcctagatcatcagttcatacaaacctccatagagttcaacatcttaaacaagaatatcctgattgctccaccatgttggcaagctgctctagccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattcgagctctaactataacggtcctaaggtagcgagcgatcgcgcgtattggctagagcagcttgccaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacacgctgaaatcaccagtctctctctacaaatctatctctgtttaaacatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctagagttcgtgaccgccgccgggatcactctcggcatggacgagctgtacaagtctgagaaggatgagctctgacggaccggatctgtcgatcgacaagctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcccccgaattagcggccgcattaccctgttatccctactttcggattataacatcacatctatgtcgggtgcggagaaagaggtaatgaaatggcatgacgatcaaccataccagataactatgactctcttaaggtagccaaataatacccggtctgaacgaggtggcaaacagctattatgggtattatgggtggtacccggggatcctctagagtcgacctgcaggcatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgctagagcagcttgagcttggatcagattgtcgtttcccgccttcagtttaaactatcagtgtttgacaggatatattggcgggtaaacctaagagaaaagagcgtttattagaataacggatatttaaaagggcgtgaaaaggtttatccgttcgtccatttgtatgtg

SEQ ID NO: 18 - DNA, 9172 bp, Artificial
  plant transformation vector
  misc_feature (1)..(9172): 133869 vector
  misc_feature (2354)..(6184): Lb6Cpf1
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggccgcatcccgggttgtagctccacgcgcttcggcaggggaagctctacttaatcggaagtccatatgtaactataacggtcctaaggtagcgagcgatcgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatactactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcacggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatactgatttttttagccctgccttcatggatgatggcatatgcagcagctatatgtgtacgctatttatttgcttggtactgtttcttttgtcgatgctcaccctgttgtttggtgttacttctgcaggtttaaacatggccccaaagaaaaagcgcaaggtgatgcacgagaacaacggcaagatcgccgataacttcatcggcatctaccccgtgtctaagacccttaggttcgagcttaagccagtgggaaagacccaagagtacatcgagaagcacggaatcctcgatgaggatcttaagcgcgctggcgattacaagtccgtgaagaagattatcgacgcctaccacaagtacttcatcgacgaggctctcaacggaatccagcttgatggactcaagaactactacgagctgtacgagaagaagcgcgacaacaacgaagagaaagagttccaaaagatccagatgtccctgcgcaagcagatcgtgaagagattctctgagcacccacagtacaagtacctcttcaagaaagaactcatcaagaacgtgctccccgagttcaccaaggataacgctgaggaacagaccctcgtgaagtccttccaagagttcactacctacttcgagggcttccaccagaaccgcaagaacatgtactccgacgaggaaaagtccaccgctatcgcttacagggttgtgcatcagaacctgccaaagtacattgacaacatgcgcatcttctccatgatcctcaacaccgacatccgctctgatctcaccgagcttttcaacaacctcaagaccaagatggacatcaccatcgtggaagagtacttcgctatcgacggcttcaacaaggtggtgaaccagaaaggcatcgacgtgtacaacaccatcctcggagctttctccaccgatgacaacaccaagatcaagggcctcaacgagtacatcaacctctacaaccagaagaacaaggctaagctccccaagctcaagccactcttcaagcagattctctccgaccgcgacaagatctccttcattccagagcagttcgactccgataccgaggttttggaggctgtggatatgttctacaaccgccttctccagttcgtgatcgagaacgagggacagattaccatctccaagctcctcaccaacttctccgcttacgacctcaacaagatctacgtgaagaacgacaccaccatctccgccatctctaacgatctcttcgacgactggtcctacatctctaaggctgtgagagagaactacgactccgagaacgtggacaagaacaagagagctgctgcctacgaagaaaagaaagagaaggccctctccaagattaagatgtactcaatcgaggaactcaacttcttcgtcaagaagtactcctgcaacgagtgccacatcgagggatacttcgagagaaggatcctcgagattctcgacaagatgaggtacgcctacgagtcctgcaagatccttcacgataagggcctgatcaacaacatcagcctttgccaagataggcaggccatctccgagttgaaggatttcctcgactccatcaaagaggtgcagtggcttcttaagccactcatgattggacaagagcaggccgacaaagaagaggccttctacactgagcttctccgcatttgggaagagttggagccaatcactctgctctacaacaaggttcgcaactacgtgaccaagaagccctacactcttgagaaggtgaagctgaacttctacaagagcaccctcctcgatggctgggataagaacaaagaaaaggacaacctcgggatcatcctcctcaaggatggacagtactacctcggcattatgaaccgccgcaacaacaagattgctgatgatgctccactcgccaagaccgacaacgtgtacagaaagatggagtacaagttgctcaccaaggtgtccgctaacctgcctaggatcttcctgaaggacaagtacaacccctccgaagagatgctcgagaagtatgagaagggaacccacctcaagggtgagaacttctgcattgatgactgccgcgagctgatcgacttcttcaaaaagggcatcaagcagtacgaggactggggccaattcgacttcaagttctctgacaccgagtcctacgatgacatctccgctttctacaaagaggttgagcaccagggctacaagattaccttcagggatatcgacgagacttacatcgacagccttgtgaacgagggtaagctctacctcttccagatctataacaaggacttctcaccatactccaagggcaccaagaaccttcacaccctttactgggagatgctgttctcccagcagaacctccagaacatcgtgtacaagctcaacggcaacgccgagatcttctaccgcaaggcttctattaaccagaaggacgtcgtcgtccacaaggccgatttgcccatcaaaaacaaggacccacagaactctaagaaagaatccatgttcgactacgacatcatcaaggacaagcgcttcacttgcgacaagtaccagttccacgtgccaatcaccatgaacttcaaggctcttggggagaaccacttcaaccgtaaggtgaacaggctcatccacgacgctgagaacatgcacatcatcggaattgatcgcggagagcgcaaccttatctacctctgcatgatcgacatgaagggcaacattgtgaagcagatctccctgaacgagatcatctcctacgacaaaaacaaactcgagcacaagcgcaactaccaccagttgcttaagaccagagaggatgagaacaagtccgctaggcaatcctggcagaccatccacactatcaaagagctgaaagagggctacctcagccaggttatccatgtgattaccgacctcatggtcgagtacaacgctattgtggtgcttgaggacctgaacttcggattcaagcaaggcaggcaaaagttcgagaggcaggtctaccagaaattcgagaagatgttgatcgataagctcaactacctggtggacaagtccaaaggcatggatgaggatggcggattgcttcacgcttaccaacttaccgacgagttcaagtctttcaagcagctcggaaagcagtccggcttcctctattacattccagcctggaacaccagcaagctcgatccaactactggattcgtgaacctcttctacaccaagtacgagtccgtcgagaagtctaaagagttcatcaacaacttcacctccatcctctacaatcaagagcgcgagtatttcgagttcctcttcgactactctgccttcacctctaaggcagagggatccagacttaagtggaccgtttgctccaagggtgaacgcgttgagacttataggaaccccaagaaaaacaacgagtgggacacccagaagatcgaccttaccttcgaactcaagaagctgttcaacgactactccatttccttgctcgatggcgatctcagggaacagatgggaaagatcgacaaggctgacttctataagaaattcatgaagcttttcgccctcatcgtccagatgaggaactctgatgagcgcgaggacaagctcatttccccagtgcttaacaagtacggcgccttcttcgaaaccggaaagaacgagagaatgccactcgatgctgatgctaacggcgcttacaacattgctaggaaaggcctctggatcatcgaaaagatcaagaacaccgacgttgagcagctcgacaaggtcaagctcaccatcagcaacaaagagtggttgcagtacgctcaagagcacatcctttgacggaccggtcatgggtcgtttaagctgccgatgtgcctgcgtcgtctggtgccctctctccatatggaggttgtcaaagtatctgctgttcgtgtcatgagtcgtgtcagtgttggtttaataatggaccggttgtgttgtgtgtgcgtactacccagaactatgacaaatcatgaataagtttgatgtttgaaattaaagcctgtgctcattatgttctgtctttcagttgtctcctaatatttgcctgcaggtactggctatctaccgtttcttacttaggaggtgtttgaatgcactaaaactaatagttagtggctaaaattagttaaaacatccaaacaccatagctaatagttgaactattagctatttttggaaaattagttaatagtgaggtagttatttgttagctagctaattcaactaacaatttttagccaactaacaattagtttcagtgcattcaaacacccccttaatgttaacgtggttctatctaccgtctcctaatatatggttgattgttcggtttgttgctatgctattgggttctgattgctgctagttcttgctgaatccagaagttctcgtagtatagctcagattcatattatttatttgagtgatggcgcgcccgagtatcgaattcctgcaggcatgcaagcgatccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcggcggccgcattaccctgttatccctaaagcttgaattcagtacattaaaaacgtccgcaatgtgttattaagttgtctaagcccgggctgggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 19 - DNA, 3095 bp, Artificial
  plant transformation vector
  misc_feature (1)..(3095): vector 133432
  misc_feature (982)..(1024): gRNA
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggccgcatcccgggttgtagctccacgcgcttcggcaggggaagctctacttaatcggaagtccatatgtaactataacggtcctaaggtagcgagcgatcgctttgtgaaagttgaattacggcatagccgaaggaataacagaatcgtttcacactttcgtaacaaaggtcttcttatcatgtttcagacgatggaggcaaggctgatcaaagtgatcaagcacataaacgcatttttttaccatgtttcactccataagcgtctgagattatcacaagtcacgtctagtagtttgatggtacactagtgacaatcagttcgtgcagacagagctcatacttgactacttgagcgattacaggcgaaagtgtgaaacgcatgtgatgtgggctgggaggaggagaatatatactaatgggccgtatcctgatttgggctgcgtcggaaggtgcagcccacgcgcgccgtaccgcgcgggtggcgctgctacccactttagtccgttggatggggatccgatggtttgcgcggtggcgttgcgggggatgtttagtaccacatcggaaaccgaaagacgatggaaccagcttataaacccgcgcgctgtagtcagcttgaatttctactgttgtagataagggaccgcagcaagctgccgggtttttttgttttgcggccgcattaccctgttatccctaaagcttgaattcagtacattaaaaacgtccgcaatgtgttattaagttgtctaagcccgggctgggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 20 - DNA, 5846 bp, Artificial
  plant transformation vector
  misc_feature (1)..(5846): vector 131592
  misc_feature (722)..(1754): hygromycin resistance gene
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggccgcatcccgggttgtagctccacgcgcttcggcaggggaagctctacttaatcggaagtccatatgatctatgtcgggtgcggagaaagaggtaatgaaatggcagcgatcgcagctgaattaacgccgaattaattcgggggatctggattttagtactggattttggttttaggaattagaaattttattgatagaagtattttacaaatacaaatacatactaagggtttcttatatgctcaacacatgagcgaaaccctataggaaccctaattcccttatctgggaactactcacacattattatggagaaactcgagcttgtcgatcgacagatcccggtcggcatctactctatttctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagtcgtggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatagcctccgcgaccggttgtagaacagcgggcagttcggtttcaggcaggtcttgcaacgtgacaccctgtgaacggcgggagatgcaataggtcaggctctcgctaaactccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggctttttcatatctcattgcccgggaagcttatcgtctacctgcagaagtaacaccaaacaacagggtgagcatcgacaaaagaaacagtaccaagcaaataaatagcgtatgaaggcagggctaaaaaaatccacatatagctgctgcatatgccatcatccaagtatatcaagatcaaaataattataaaacatacttgtttattataatagataggtactcaaggttagagcatatgaatagatgctgcatatgccatcatgtatatgcatcagtaaaacccacatcaacatgtatacctatcctagatcgatatttccatccatcttaaactcgtaactatgaagatgtatgacacacacatacagttccaaaattaataaatacaccaggtagtttgaaacagtattctactccgatctagaacgaatgaacgaccgcccaaccacaccacatcatcacaaccaagcgaacaaaaagcatctctgtatatgcatcagtaaaacccgcatcaacatgtatacctatcctagatcgatatttccatccatcatcttcaattcgtaactatgaatatgtatggcacacacatacagatccaaaattaataaatccaccaggtagtttgaaacagaattctactccgatctagaacgaccgcccaaccagaccacatcatcacaaccaagacaaaaaaaagcatgaaaagatgacccgacaaacaagtgcacggcatatattgaaataaaggaaaagggcaaaccaaaccctatgcaacgaaacaaaaaaaatcatgaaatcgatcccgtctgcggaacggctagagccatcccaggattccccaaagagaaacactggcaagttagcaatcagaacgtgtctgacgtacaggtcgcatccgtgtacgaacgctagcagcacggatctaacacaaacacggatctaacacaaacatgaacagaagtagaactaccgggccctaaccatggaccggaacgccgatctagagaaggtagagagggggggggggggaggacgagcggcgtaccttgaagcggaggtgccgacgggtggatttgggggagatctggttgtgtgtgtgtgcgctccgaacaacacgaggttggggaaagagggtgtggagggggtgtctatttattacggcgggcgaggaagggaaagcgaaggagcggtgggaaaggaatcccccgtagctgccgtgccgtgagaggaggaggaggccgcctgccgtgccggctcacgtctgccgctccgccacgcaatttctggatgccgacagcggagcaagtccaacggtggagcggaactctcgagaggggtccagaggcagcgacagagatgccgtgccgtctgcttcgcttggcccgacgcgacgctgctggttcgctggttggtgtccgttagactcgtcgacggcgtttaacaggctggcattatctactcgaaacaagaaaaatgtttccttagtttttttaatttcttaaagggtatttgtttaatttttagtcactttattttattctattttatatctaaattattaaataaaaaaactaaaatagagttttagttttcttaatttagaggctaaaatagaataaaatagatgtactaaaaaaattagtctataaaaaccattaaccctaaaccctaaatggatgtactaataaaatggatgaagtattatataggtgaagctatttgcaaaaaaaaaggagaacacatgcacactaaaaagataaaactgtagagtcctgttgtcaaaatactcaattgtcctttagaccatgtctaactgttcatttatatgattctctaaaacactgatattattgtagtagtatagattatattattcgtagagtaaagtttaaatatatgtataaagatagataaactgcacttcaaacaagtgtgacaaaaaaaatatgtggtaattttttataacttagacatgcaatgctcattatctctagagaggggcacgaccgggtcacgctgcactgcagaagcttgctggcggccgcctatgactctcttaaggtagccaaataagcttgaattcagtacattaaaaacgtccgcaatgtgttattaagttgtctaagcccgggctgggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

(19) United States
(12) Patent Application Publication
(10) Pub. No.: US 2023/0235344 A1
(43) Pub. Date: Jul. 27, 2023

(54) PLANT CELL TREATMENTS TO IMPROVE PLANT TRANSFORMATION

(71) Applicant: BENSON HILL, INC., St. Louis, MO (US)

(72) Inventors: Diaa Alabed, Creve Coeur, MO (US); Lorena Beatriz Moeller, Wildwood, MO (US)

(73) Assignee: BENSON HILL, INC., St. Louis, MO (US)

(21) Appl. No.: 18/002,175

(22) PCT Filed: Jun. 24, 2021
(86) PCT No.: PCT/IB2021/055634

§ 371 Date: Dec. 16, 2022

Related U.S. Application Data
(60) Provisional application No. 63/043,487, filed on Jun. 24, 2020.

(51) Int. Cl. C12N 15/82; C12N 9/22

(52) U.S. Cl. CPC C12N 15/8213; C12N 9/22; C12N 15/8207; C12N 15/8205; C12N 2310/20

(57) ABSTRACT

Provided herein are methods for increasing plant cell transformation efficiency. These methods include exposing the plant cells to a liquid medium containing a surfactant. Following exposure to the surfactant-containing medium, the cells can become more amenable to transformation and may be genetically transformed using methods known in the art. Exposure of the cells to the surfactant-containing medium prior to transformation can increase plant transformation efficiency when compared to transformation efficiency of cells not exposed to the surfactant-containing medium.

### FIELD OF THE INVENTION

The present invention relates to compositions and methods for treating plant cells in such a way that they are more amenable to genetic transformation than untreated plant cells.

### REFERENCE TO A SEQUENCE LISTING SUBMITTED AS A TEXT FILE VIA EFS-WEB

The official copy of the sequence listing is submitted concurrently with the specification as a text file via EFS-Web, in compliance with the American Standard Code for Information Interchange (ASCII), with a file name of B88552_1290US_P1_Seq_List_6-24-20.txt, a creation date of Jun. 24, 2020, and a size of 126 Kb. The sequence listing filed via EFS-Web is part of the specification and is hereby incorporated in its entirety by reference herein.

### BACKGROUND OF THE INVENTION

Plant transformation generally encompasses protocols for the introduction of one or more plant-expressible foreign gene(s) into plant cells. After this introduction, plants may be regenerated from the cell(s) into which foreign gene(s) have been introduced such that fertile progeny plants may be obtained which stably maintain and express the foreign gene. More recently developed plant transformation protocols make use of so-called “genome editing” technologies that allow for the insertion of foreign genetic material at pre-determined genomic loci, precise modification of DNA sequences at pre-determined genomic loci, and/or deletion of DNA sequences from pre-determined genomic loci. Numerous plant species have been transformed using standard transformation techniques and/or genome editing techniques. Transgenic and/or genome edited agronomic crops, as well as fruits and vegetables, are of commercial interest. Such crops include but are not limited to maize, rice, soybeans, canola, sunflower, alfalfa, sorghum, wheat, cotton, peanuts, tomatoes, potatoes, peas, and the like.

Thus, the methods of this disclosure can be used to provide transformed plants with combinations of traits that may provide benefits to growers, processors, and consumers. Methods for increasing plant transformation efficiency are provided.

### SUMMARY OF THE INVENTION

Methods of improving transformation efficiencies of plant cells or plant tissues by exposing them to surfactants are described herein. Various ways of exposing plants to surfactants can be used in conjunction with a wide range of plant transformation protocols, including, but not limited to, infection-mediated transformation, for example, DNA transfer to plant cells by bacteria (e.g. Agrobacterium tumefaciens), virus-mediated transformation, biolistics (e.g. gene-gun/microprojectile transfer), electroporation, and/or other transformation methods. In certain embodiments, plant cells are exposed to a surfactant to improve the efficiency of delivering biologically active materials other than nucleic acids (e.g. proteins) into the plant cells. Other methods enabling the delivery of biologically active materials into plant cells are also described. Moreover, the plant cells can be exposed to the surfactant or surfactant-containing medium at any stage before or after transformation.

### DETAILED DESCRIPTION OF THE INVENTION

Methods to increase the transformation frequency and/or efficiency in plant cells by exposing the plant material to be transformed to a surfactant are described. The methods include exposing plant cells or tissues to a liquid medium containing a surfactant, then removing the surfactant-containing medium, then transforming the plant cells or tissues by a wide range of transformation methods. “Pre-conditioning” or “preconditioning” is the exposure of the plant cells to a surfactant-containing medium for a period lasting between 5 minutes and 90 minutes, followed by removal of the surfactant containing medium prior to transformation of the cells that were exposed to a surfactant-containing medium. Pre-conditioning and other methods of exposing plant cells to a surfactant result in improved transient expression of introduced genes, enhanced production of stably transformed cells and sectors, and improved recovery of regenerated transformed plants.

“Plant cells or tissues” includes, without limitation, seeds (whole and partial), cells, callus, embryos, leaf discs, hypocotyl tissue, hairy roots, cotyledons, immature and mature embryos, flowers, reproductive organs and other plant cells and tissues that are suitable for transformation using the methods of the invention.

Transformation of plant cells requires the introduction of the transforming DNA, for example and without limitation, by contacting the plant cells with a suitable strain of Agrobacterium that harbors one or more transformation plasmids. Strains of Agrobacterium differ from one another in their ability to transform plant cells of various species. Regardless of the combination of Agrobacterium strain/host plant considered, Agrobacterium acts through attachment to the host cell during transformation. See McCullen and Binns, 2006, Ann. Rev. Cell. Dev. Biol. 22:101-127; and Citovsky et al., 2007, Cell. Microbiol. 9:9-20. For this reason, methods that affect plant cell wall structure and/or the ability of plant cells to take in material such as genetic material including DNA, such as those disclosed herein using surfactants (Buchanan 1965 Iowa State University Dissertation), may produce increases in transformation efficiency.

As described herein, a plant refers to a whole plant, any part thereof, or a cell or tissue culture derived from a plant, comprising any of: whole plants, plant components or organs (e.g., leaves, stems, roots, etc.), plant tissues, seeds, plant cells, protoplasts and/or progeny of the same. A plant cell is a biological cell of a plant, taken from a plant or derived through culture of a cell taken from a plant.

As used herein, a “mutation” is any change in a nucleic acid sequence. Nonlimiting examples comprise insertions, deletions, duplications, substitutions, inversions, and translocations of any nucleic acid sequence, regardless of how the mutation is brought about and regardless of how or whether the mutation alters the functions or interactions of the nucleic acid. For example and without limitation, a mutation may produce altered enzymatic activity of a ribozyme, altered base pairing between nucleic acids (e.g. RNA interference interactions, DNA-RNA binding, etc.), altered mRNA folding stability, and/or how a nucleic acid interacts with polypeptides (e.g. DNA-transcription factor interactions, RNA-ribosome interactions, gRNA-endonuclease reactions, etc.).

A mutation might result in the production of proteins with altered amino acid sequences (e.g. missense mutations, nonsense mutations, frameshift mutations, etc.) and/or the production of proteins with the same amino acid sequence (e.g. silent mutations). Certain synonymous mutations may create no observed change in the plant while others that encode for an identical protein sequence nevertheless result in an altered plant phenotype (e.g. due to codon usage bias, altered secondary protein structures, etc.). Mutations may occur within coding regions (e.g., open reading frames) or outside of coding regions (e.g., within promoters, terminators, untranslated elements, or enhancers), and may affect, for example and without limitation, gene expression levels, gene expression profiles, protein sequences, and/or sequences encoding RNA elements such as tRNAs, ribozymes, ribosome components, and microRNAs.

Methods disclosed herein are not limited to mutations made in the genomic DNA of the plant nucleus. For example, in certain embodiments a mutation is created in the genomic DNA of an organelle (e.g. a plastid and/or a mitochondrion). In certain embodiments, a mutation is created in extrachromosomal nucleic acids (including RNA) of the plant, cell, or organelle of a plant. Nonlimiting examples include creating mutations in supernumerary chromosomes (e.g. B chromosomes), plasmids, and/or vector constructs used to deliver nucleic acids to a plant. It is anticipated that new nucleic acid forms will be developed and yet fall within the scope of the claimed invention when used with the teachings described herein.

Methods disclosed herein are not limited to certain techniques of mutagenesis. Any method of creating a change in a nucleic acid of a plant can be used in conjunction with the disclosed invention, including the use of chemical mutagens (e.g. methanesulfonate, sodium azide, aminopurine, etc.), genome/gene editing techniques (e.g. CRISPR-like technologies, TALENs, zinc finger nucleases, and meganucleases), ionizing radiation (e.g. ultraviolet and/or gamma rays) temperature alterations, long-term seed storage, tissue culture conditions, targeting induced local lesions in a genome, sequence-targeted and/or random recombinases, etc. It is anticipated that new methods of creating a mutation in a nucleic acid of a plant will be developed and yet fall within the scope of the claimed invention when used with the teachings described herein.

Similarly, the embodiments disclosed herein are not limited to certain methods of introducing nucleic acids into a plant and are not limited to certain forms or structures that the introduced nucleic acids take. Any method of transforming a cell of a plant described herein with nucleic acids are also incorporated into the teachings of this innovation.

Methods disclosed herein are not limited to any size of nucleic acid sequences that are introduced, and thus one could introduce a nucleic acid comprising a single nucleotide (e.g. an insertion) into a nucleic acid of the plant and still be within the teachings described herein. Nucleic acids introduced in substantially any useful form, for example, on supernumerary chromosomes (e.g. B chromosomes), plasmids, vector constructs, additional genomic chromosomes (e.g. substitution lines), and other forms is also anticipated. It is envisioned that new methods of introducing nucleic acids into plants and new forms or structures of nucleic acids will be discovered and yet fall within the scope of the claimed invention when used with the teachings described herein.

A plant refers to a whole plant, any part thereof, or a cell or tissue culture derived from a plant, comprising any of: whole plants, plant components or organs (e.g., leaves, stems, roots, etc.), plant tissues, seeds, plant cells, protoplasts and/or progeny of the same. A plant cell is a biological cell of a plant, taken from a plant or derived through culture of a cell taken from a plant.

As used herein, a seed is a fertilized ovule containing a plant embryo. In angiosperms, the seed is covered by a protective layer called the seed coat. It is a known challenge in the art of plant transformation that the seed coat forms a barrier to delivering molecules of interest to the embryo. Consequently, methods known in the art of transforming plants require that the seed coat be broken and/or removed entirely. Improvements of the methods taught herein include transforming a plant embryo while the embryo remains inside a substantially intact seed, e.g. a whole seed. Embodiments disclosed herein include methods of transforming a plant that require essentially little to no manipulation of the seed coat, or the cotyledons, hypocotyl, radicle of the embryo by exposing the seed to a surfactant composition as disclosed elsewhere herein.

In certain embodiments, the seed coat may be perforated, scored, abraded or otherwise or wounded to help the delivery of a molecule of interest to the embryo, for example, by the use of a tool that can damage the seed coat. In certain embodiments, this can be accomplished by subjecting the seeds to a sonication treatment to create small holes in the seed coat of the whole seed. As used herein, the term whole seed includes a seed that has its seed coat connected to the seed. In specific embodiments, the whole seed has been subjected to a sonication step but still has its seed coat connected to the seed.

It is expected that the teachings herein can be combined with other methods of the art that require the seed coat is substantially damaged, i.e. that the seed coat is been cut or ripped open and even removed to expose the embryo within. However, the concept that it is possible to reliably transform an embryo without creating an explant by removing the embryo from the seed or cutting away parts of the seed is an important teaching herein.

A population means a set comprising any number, including one, of individuals, objects, or data from which samples are taken for evaluation, e.g. estimating QTL effects and/or disease tolerance. Most commonly, the terms relate to a breeding population of plants from which members are selected and crossed to produce progeny in a breeding program. A population of plants can include the progeny of a single breeding cross or a plurality of breeding crosses and can be either actual plants or plant derived material, or in silico representations of plants. The member of a population need not be identical to the population members selected for use in subsequent cycles of analyses nor does it need to be identical to those population members ultimately selected to obtain a final progeny of plants. Often, a plant population is derived from a single biparental cross but can also derive from two or more crosses between the same or different parents. Although a population of plants can comprise any number of individuals, those of skill in the art will recognize that plant breeders commonly use population sizes ranging from one or two hundred individuals to several thousand, and that the highest performing 5-20% of a population is what is commonly selected to be used in subsequent crosses to improve the performance of subsequent generations of the population in a plant breeding program.

Crop performance is used synonymously with plant performance and refers to of how well a plant grows under a set of environmental conditions and cultivation practices. Crop performance can be measured by any metric a user associates with a crop's productivity (e.g. yield), appearance and/or robustness (e.g. color, morphology, height, biomass, maturation rate), product quality (e.g. fiber lint percent, fiber quality, seed protein content, seed carbohydrate content, etc.), cost of goods sold (e.g. the cost of creating a seed, plant, or plant product in a commercial, research, or industrial setting) and/or a plant's tolerance to disease (e.g. a response associated with deliberate or spontaneous infection by a pathogen) and/or environmental stress (e.g. drought, flooding, low nitrogen or other soil nutrients, wind, hail, temperature, day length, etc.). Crop performance can also be measured by determining a crop's commercial value and/or by determining the likelihood that a particular inbred, hybrid, or variety will become a commercial product, and/or by determining the likelihood that the offspring of an inbred, hybrid, or variety will become a commercial product. Crop performance can be a quantity (e.g. the volume or weight of seed or other plant product measured in liters or grams) or some other metric assigned to some aspect of a plant that can be represented on a scale (e.g. assigning a 1-10 value to a plant based on its disease tolerance).

Methods previously disclosed in the art describe that Agrobacterium-mediated transformation efficiencies can be improved by exposing the infecting bacteria to a surfactant prior to infection. However, the published art attributes the benefits of using surfactants to the effects the surfactant has on the Agrobacterium and/or the ability of the Agrobacterium to adhere or contact the plant cells and, consequently, only instruct users to first culture the Agrobacterium in a surfactant-containing medium prior to exposing the surfactant and/or Agrobacterium to the plant cell, i.e. prior to infection and/or include surfactants “in the infection medium”.

International patent application No. PCT/IB2019/061126, incorporated herein by reference as the '126 application, revealed the surprising discovery that it is unnecessary to expose Agrobacterium to surfactant. Among other innovations, those teachings reveal that exposing only the plant cells to surfactant and then removing the plant cells from the surfactant before exposing the plant cells to the infection medium containing the transforming Agrobacteria still produced significant improvement in transformation efficiencies. The discoveries suggest that the benefits of using surfactant are due largely to the effects the surfactant has on the plant cells and not necessarily the Agrobacteria.

Although the actual mechanism of the surfactant-plant cell interaction is not required to make or use the innovations described in the teachings of the '126 application, it is useful to posit a new hypothesis that the benefits of using a surfactant are largely specific to its effect on the plant cells, for example, that the effects are due largely to the surfactant altering gene expression in the plant cells, changing the properties of plant cell membranes and/or cell walls, making them more amenable to the transfer of genetic information into the plant cell. It is also possible that there are synergistic effects on plant cell health, promoted by the surfactant enabling better absorption of media components which results in enhanced regeneration, selection and transformation efficiency. This is consistent with the teachings herein revealing that improved transformation efficiencies can be achieved when surfactants are not present during the inoculation step of Agrobacterium-mediated transformation.

In contrast to the art, the '126 application teachings are not limited to Agrobacterium mediated transformation. If the improvement in transformation efficiencies does not require the surfactant to interact with the infecting bacteria, or be involved in bacterial—plant cell interactions during infection, then it follows that surfactants will improve transformation efficiencies when used in conjunction with methods that are not based on infection, for example, biolistics.

The '126 application reveals for the first time that surfactants can be used to improve transformation efficiencies that are not based on infection, for example, by incorporating surfactants in biolistic transformation protocols. This discovery opens a new world of potential embodiments, including at least the use of surfactants during biolistic transformation to dramatically improve genome editing, which are enabled in Example 17 of the '126 application.

Since treating plant cells to a surfactant improves non-bacterial-mediated transformation methods (physical or chemical methods), like biolistics, it follows that it will also improve transformation efficiencies for other non-bacterial-mediated transformation methods where genetic information must pass the plant cell membrane and/or plant cell wall, for example, electroporation transformation. The teachings of the '126 application and/or those herein reveal how this can be done.

Disclosed herein are improved methods consistent with the teachings of the '126 application wherein surfactant-based improvements in plant transformation are realized without incubating or culturing an infecting bacterium in surfactant prior to exposure to the plant cells. In certain embodiments, a user of these innovations can expose plant cells targeted for transformation to Agrobacterium (or some other infecting bacterium or virus) before they are exposed to a surfactant and still achieve improved transformation efficiencies.

For example, one step in the bacteria-mediated infection transformation process that follows infection is coculture, during which the bacterium and plant cells are grown together to give the bacterium adhering to the plant cells time to transfer genetic material into the plant cells following an infection step. Experiments disclosed herein demonstrate how plant cells can be exposed to surfactant only during or after the coculture step (e.g during selection and regeneration steps following transformation) to achieve improved transformation efficiencies.

Experiments disclosed herein also demonstrate that improved transformation efficiencies can be achieved when plants are only exposed to surfactants during an initial seed imbibition step, for example, before soybean explants are removed from their seeds and prepared for infection. Moreover, disclosed herein are methods for transforming conditioning whole seeds with the disclosed surfactants without the need for removal of explants or disturbance of the seed coat.

Without being limited by theory, increases in plant transformation efficiency by the methods disclosed herein may result from the ability of surfactants to modify plant cell walls, allowing for more efficient introduction of DNA into the plant cells. One may therefore utilize the chemical differences between different surfactant agents to promote plant cell wall modifications so that enhanced transformation efficiencies may be observed.

Surfactants belong to several chemical classes, and one skilled in the field of plant transformation will understand that different chemical classes of surfactants may be used to enhance plant transformation efficiency with different plant hosts. Examples of surfactants from these chemical classes useful with the methods disclosed herein include adjuvants, non-ionic surfactants, anionic surfactants, oil-based surfactants, amphoteric surfactants, and polymeric surfactants. An example of a preferred surfactant useful with the methods described herein is a non-ionic trisiloxane surfactant such as BREAK-THRU® S233 surfactant from Evonik Industries (Essen, Germany). Examples of further preferred surfactants useful with the methods described herein include trisiloxane alkoxylates, ethoxylated soybean oils, alcohol ethoxylate C-13s, C2-Ct4-alkyldimethyl betaines, and di-sec-butylphenol ethylene oxide-propylene oxide block co-polymers. Additional surfactants of various chemical types that may be used to practice the methods described herein are disclosed in U.S. patent application Ser. No. 13/715,118. In specific embodiments, the surfactant disclosed herein is Break-Thru S233, Break-Thru 5240, Break-Thru S279, Break-Thru S301, or Pluronic F-68.

The methods disclosed herein utilize the transformation-enhancing properties of surfactants to dramatically increase transformation efficiency in plants such as immature maize embryos, for example, by Agrobacterium (e.g., Agrobacterium tumefaciens). The surfactants used with the methods described herein are selected, as suggested above, based upon the ability to modify plant cell wall properties in such a way that will enhance transformation efficiency. In order to improve transformation efficiency, plant cells are exposed to an effective amount of surfactant. As used herein, an “effective amount” of a surfactant refers to an amount of surfactant that improves the transformation efficiency of a plant, plant cell, or population thereof. In some embodiments, the effective amount of a surfactant increases transformation efficiency of a plant, plant cell, or population thereof by at least 10%, at least 20%, at least 30%, at least 40%, at least 50%, at least 60%, at least 70%, at least 80%, at least 90%, at least 99% or more when compared to the transformation efficiency in the absence of the surfactant. The concentration of surfactant in the liquid medium can be 0.0001-0.1% (v/v), 0.001-0.1% (v/v), 0.001-0.09% (v/v), 0.001-0.08% (v/v), 0.001-0.07% (v/v), 0.001-0.06% (v/v), 0.001-0.05% (v/v), 0.01-0.04% (v/v), 0.001-0.03% (v/v), or 0.001-0.2% (v/v). Methods for measuring transformation efficiency are known in the art and described herein below.

In some embodiments, the plant, plant cell, seed, or other plant part is exposed to a surfactant from between 1 min to 4 hrs. For example, exposure could be for 5 min to 2 hours, 5 min-90 min, 5-60 min, 5-30 min, or any time sufficient to increase the transformation efficiency. In particular embodiments, the exposure to a surfactant or surfactant containing medium lasts between 5-60 min.

One or more additional surfactants can also be used with the methods described herein. As indicated, the transformation efficiency is dependent on a variety of factors including plant species and/or tissue-type and/or strain of infecting bacterium. Using routine methods, it is customary to adjust certain variables that are known to improve biolistic transformation efficiencies under different conditions. For instance, the properties of the particle used to bombard the cells is an important variable, for example, particle composition (i.e. which elements, e.g. tungsten, gold, etc., or combination thereof), and that mass, density, volume and the velocity to which the particle/particles is/are accelerated during bombardment can be tested to determine the variable threshold combinations that are more successful, and may be specific to certain cell types within the same plant and even thresholds specific to cells between different genotypes of closely-related germplasm. Optimal thresholds of other factors are commonly determined through routine testing, including the types and/or concentration of chemicals that are employed in the process (e.g. calcium chloride, Trans-IT, spermidine) and the amount of nucleic acids and/or ribonucleic proteins and can be combined with the teachings herein to improve transformation efficiencies under a broad range of conditions.

Given the variety of interactions involved, a system comprising two or more surfactants and/or exposing plant cells to surfactants at multiple steps in the transformation process to provide enhanced transformation efficiency is also envisioned.

In some embodiments, the cells or tissues are exposed to the surfactant containing medium for a length of time between 5 minutes and 90 minutes. In some embodiments, the cells or tissues are exposed to the surfactant containing medium for a length of time between 5 minutes and 60 minutes. In certain embodiments, after exposure to the surfactant containing medium, the surfactant containing medium is removed by pipetting or other suitable methods that result in the removal of substantially all the preconditioning medium and the cells or tissues are resuspended in a medium that lacks surfactant. In other embodiments, the transformed tissue is exposed to surfactant containing medium for the duration of the tissue culture step, ranging from 1 day to 3 weeks (co-culture and selection).

The methods of the invention can be used with any plant transformation protocol. Many plant transformation methods are known in the art, and for obtaining plants that stably maintain and express the introduced gene. Such techniques include biolistic transformation (e.g., U.S. Pat. Nos. 4,945,050; 5,141,131), WHISKERS™ technology (see, e.g., U.S. Pat. Nos. 5,302,523; 5,464,765), electroporation technology (e.g., WO 87/06614, U.S. Pat. Nos. 5,472,869, 5,384,253, WO 92/09696, and WO 93/21335), fusion of plant protoplasts with liposomes containing the DNA to be delivered, direct injection of the DNA, and introduction of foreign DNA through the use of suitable bacteria including Agrobacterium sp., Ensifer sp, (e.g. E. adhaerens)., Ochrobactrum sp. (e.g. O. harardense), Rhizobium sp. (e.g. R. etli), and/or the collective of bacterial strains termed Transbacter™. Teachings herein are also useful for improving the plant transformation efficiencies of other bacteria, including new strains yet to be discovered and/or published.

Methods described herein for transforming plants include certain standard steps known in the art. Certain methods include the steps of imbibition, wherein seeds are allowed to imbibe in different solutions, usually for a period of several hours or even days, followed by the step of explant preparation, wherein the seed coat and other tissues are removed to expose cells of the target tissue that are amenable to transformation, followed by infection, wherein the explants are exposed to a culture of bacteria capable of transforming plant cells (e.g. Agrobacteria), followed by coculture, during which the explants and the infecting bacteria adhering to the plant cells are grown together to facilitate delivery of nucleic acids into the plant cells, followed by the various steps of transgenic selection and recovery, including callus induction, shoot elongation, regeneration, and/or rooting, etc. As used herein, the term imbibe refers to the exposure of the seed or other plant part to a solution for a given period of time. In specific embodiments, the seed is imbibed (i.e., exposed) to a solution comprising a surfactant in order to increase the uptake of a polynucleotide or polypeptide or increase the transformation efficiency.

The teachings herein are not limited to infection-mediated transformation systems that rely on bacteria. For example, conditioning plant cells with surfactants prior to, during, or after infection by viruses is also described. Example viruses commonly used for transformation that can be useful with these innovations include, but are not limited to, the tobacco mosaic virus, potato virus X, barley stripe mosaic virus, alfalfa mosaic virus, and geminivirus vectors. Conditioning plant cells with surfactants to make them more amenable to transformation by other viruses is also envisioned, as is using a combination of bacterial and viral transformation techniques in combination with surfactant conditioning of plant cells. See, for example, Awram P, et al. Adv Virus Res 2002, 58:81-124; Pogue G P, et. al. Annu Rev Phytopathol 2002, 40:45-74; Porta C, et. al., Biotechnol Genet EngRev 2002, 19:245-291; Scholthof K B, et. al., Genet Eng 2002, 24:67-85.

Several techniques are known for performing genome editing in plant cells including CRISPR-based genome editing techniques (for example, CRISPR nucleases include, but are not limited to, Cas9, Cpf1/Cas12a, Cms1/Cas12f, C2cl, C2c3, CasX, CasY, or other suitable CRISPR/Cas nuclease systems), meganucleases, TALENs, zinc finger nucleases (ZFNs), and other techniques. Methods described herein can be used to deliver polynucleotide sequences encoding these and/or other genome editing proteins into plant cells. Methods disclosed herein can also be used to deliver one or more genome editing proteins into plant cells where they can create edits in one or more nucleotide sequences of the plant cell without necessarily having to be expressed from a polynucleotide inside the plant cell. In some embodiments a repair donor template may be included along with the nuclease system(s) for genome editing of plant cells.

Once the inserted DNA has been integrated into the plant genome or the desired genome editing has been performed, these DNA sequence changes are usually stable throughout subsequent generations. The transformed cells grow inside the plants in the usual manner. They can form germ cells and transmit the DNA change(s) to progeny plants. Such plants can be grown in the normal manner and may be crossed with plants that have the same transformed hereditary factors or other hereditary factors. The resulting hybrid individuals have the corresponding phenotypic properties, for example, the ability to control the feeding of plant pest insects.

A number of alternative techniques can also be used for inserting DNA into a host plant cell and/or for delivering DNA that encodes nuclease(s) that can be used for genome editing (e.g., meganucleases, ZFNs, TALENs, and/or suitable CRISPR nucleases with guide RNA(s)). Those techniques include, but are not limited to, transformation with T-DNA delivered by Agrobacterium tumefaciens or Agrobacterium rhizogenes as the transformation agent and/or transformation with suitable species that may include Rhizobium, Sinorhizobium, Ochrobactrum and/or Ensifer species (see, e.g., U.S. Ser. No. 15/756,023; U.S. Pat. No. 7,888,552; WO2007/137075; WO2014/157541, WO 2006/004914). Plants may be transformed using Agrobacterium technology, as described, for example, in U.S. Pat. Nos. 5,177,010, 5,104,310, European Patent Application No. 0131624B1, European Patent Application No. 120516, European Patent Application No. 159418B1, European Patent Application No. 176112, U.S. Pat. Nos. 5,149,645, 5,469,976, 5,464,763, 4,940,838, 4,693,976, European Patent Application No. 116718, European Patent Application No. 290799, European Patent Application No. 320500, European Patent Application No. 604662, European Patent Application No. 627752, European Patent Application No. 0267159, European Patent Application No. 0292435, U.S. Pat. Nos. 5,231,019, 5,463,174, 4,762,785, 5,004,863, 5,159,135, and U.S. patent application Ser. No. 15/501,916. The use of T-DNA-containing vectors for the transformation of plant cells has been intensively researched and sufficiently described in European Patent Application 120516; An et al., (1985, EMBO J. 4:277-284); Fraley et al., (1986, Crit. Rev. Plant Sci. 4:1-46), and Lee and Gelvin (2008, Plant Physiol. 146:325-332), and is well established in the field.

A critical first step in the transformation of plant cells by Agrobacterium spp. or other suitable bacterial species for the transfer of DNA into plant cells is close contact, binding, or adherence of the bacterial cells to the cells of the host plant to be transformed. After cell-cell binding, the biology of T-DNA transfer from Agrobacterium to plant cells is known. See, e.g., Gelvin, 2003, Microbiol. Molec. Biol. Rev. 67:16-37; and Gelvin, 2009, Plant Physiol. 150:1665-1676. Without being limited by theory, transfer of T-DNA from other bacterial species may follow similar mechanisms to those understood to occur in Agrobacterium sp. At minimum, at least a T-DNA right border repeat, but often both the right border repeat and the left border repeat of the Ti or Ri plasmid will be joined as the flanking region of the gene(s) desired to be inserted into the recipient plant cell's genome. The left and right T-DNA border repeats are crucial cis-acting sequences required for T-DNA transfer. Typically, left and right T-DNA border repeats are derived from naturally occurring plasmids derived from Agrobacterium species, but suitable synthetic T-DNA border sequences (sometimes referred to as P-DNA sequences) may also be used (see, e.g., Rommens et al. (2005) Plant Physiol 139:1338-1349; U.S. Pat. Nos. 7,250,554; 7,534,934; 7,601,536; 7,619,138; 7,880,057). Various trans-acting components are encoded within the total Agrobacterium genome. Primary amongst these are the proteins encoded by the vir genes, which are normally found as a series of operons on the Ti or Ri plasmids. Various Ti and Ri plasmids differ somewhat in the complement of vir genes, with, for example, virF not always being present.

Proteins encoded by vir genes perform many different functions, including recognition and signaling of plant cell/bacteria interaction, induction of vir gene transcription, formation of a Type IV secretion channel, recognition of T-DNA border repeats, formation of T-strands, transfer of T-strands to the plant cell, import of the T-strands into the plant cell nucleus, and integration of T-strands into the plant nuclear chromosome, to name but a few. See, e.g., Tzfira and Citovsky, 2006, Curr. Opin. Biotechnol. 17:147-154.

If bacteria are used for transformation, the DNA to be inserted into the plant cell can be cloned into special plasmids, for example, either into an intermediate (shuttle) vector or into a binary vector. Intermediate vectors are not capable of independent replication in Agrobacterium cells but can be manipulated and replicated in common Escherichia co/i molecular cloning strains. It is common that such intermediate vectors comprise sequences, framed by the right and left T-DNA border repeat regions, that may include, e.g., a selectable marker gene functional for the selection of transformed plant cells, a cloning linker, cloning polylinker, or other sequence that can function as an introduction site for genes destined for plant cell transformation. Cloning and manipulation of genes desired to be transferred to plants can thus be easily performed by standard molecular biology techniques in E. coli cells, using the shuttle vector as a cloning vector. The shuttle vector can subsequently be introduced into suitable Agrobacterium plant transformation strains, or suitable strains of alternative bacterial species that may be used for plant transformation, for further work. The intermediate vector can be transferred into Agrobacterium or into the cells of other suitable bacterial species that may be used for plant transformation by means of a helper plasmid (via bacterial conjugation), by electroporation, by chemically mediated direct DNA transformation, or by other methods. Shuttle vectors can be integrated into the Ti or Ri plasmid or derivatives thereof by homologous recombination owing to sequences that are homologous between the Ti or Ri plasmid, or derivatives thereof, and the intermediate plasmid. This homologous recombination (i.e. plasmid integration) event thereby provides a means of stably maintaining the altered shuttle vector in Agrobacterium, with an origin of replication and other plasmid maintenance functions provided by the Ti or Ri plasmid portion of the co-integrant plasmid. The Ti or Ri plasmid also comprises the vir regions comprising vir genes necessary for the transfer of the T-DNA. It is common that the plasmid carrying the vir region is a mutated Ti or Ri plasmid (helper plasmid) from which the T-DNA region, including the right and left T-DNA border repeats, have been deleted, though this plasmid may also be fully synthetic. Such pTi-derived plasmids, having functional vir genes and lacking all or substantially all of the T-region and associated elements are descriptively referred to herein as helper plasmids.

The superbinary system is a specialized example of the shuttle vector/homologous recombination system (reviewed by Komari et al., 2006, In: Methods in Molecular Biology (K. Wang, ed.) No. 343: Agrobacterium Protocols, pp. 15-41; and Komori et al., 2007, Plant Physiol. 145:1155-1160). Strain LBA4404(pSB1) harbors two independently-replicating plasmids, pAL4404 and pSB1. pAL4404 is a Ti-plasmid-derived helper plasmid which contains an intact set of vir genes (from Ti plasmid pTiACH5), but which has no T-DNA region (and thus no T-DNA left and right border repeat sequences). Plasmid pSB1 supplies an additional partial set of vir genes derived from pTiBo542; this partial vir gene set includes the virB operon and the virC operon, as well as genes virG and virD1. One example of a shuttle vector used in the superbinary system is pSB 11, which contains a cloning polylinker that serves as an introduction site for genes destined for plant cell transformation, flanked by Right and Left T-DNA border repeat regions. Shuttle vector pSB11 is not capable of independent replication in Agrobacterium, but is stably maintained as a co-integrant plasmid when integrated into pSB1 by means of homologous recombination between common sequences present on pSB1 and pSB11. Thus, the fully modified T-DNA region introduced into LBA4404(pSB1) on a modified pSB11 vector is productively acted upon and transferred into plant cells by Vir proteins derived from two different Agrobacterium Ti plasmid sources (pTiACH5 and pTiBo542). The Agrobacterium tumefaciens host strain employed with the superbinary system is LBA4404(pSB1). The superbinary system has proven to be particularly useful in transformation of monocot plant species. See Hiei et al., (1994) Plant J. 6:271-282; and Ishida et al., (1996) Nat. Biotechnol. 14:745-750.

In addition to the vir genes harbored by Agrobacterium Ti plasmids, other, chromosomally-borne virulence controlling genes (termed chv genes) are known to control certain aspects of the interactions of Agrobacterium cells and plant cells, and thus affect the overall plant transformation frequency (Pan et al., 1995, Molec. Microbiol. 17:259-269). Several of the chromosomally-borne genes required for virulence and attachment are grouped together in a chromosomal locus spanning 29 kilobases (Matthysse et al., 2000, Biochim. Biophys. Acta 1490:208-212). In specific embodiments, the delivery of ribonucleotprotein complexs (RNPs) for DNA cleavage (e.g., genome editing), can be performed, for example, by particle bombardment, PEG transformation, or any other means known in the art to introduce DNA, RNA, or protein into a plant cell.

In addition to numerous technologies for transforming plants, the type of tissue which is contacted with the foreign genes may vary as well. Such tissue may include, but is not limited to, embryogenic tissue, callus tissue types I and II, hypocotyl, and meristem tissues. Almost all plant tissues may be transformed during dedifferentiation using appropriate techniques understood by a person of ordinary skill in the art. One of ordinary skill in the field of plant transformation will understand that multiple methodologies are available for the production of transformed plants, and that they may be modified and specialized to accommodate biological differences between various host plant species or plant tissues. Plant explants (for example, pieces of leaf, segments of stalk, meristems, roots, protoplasts and/or suspension-cultivated cells) can advantageously be cultivated with suitable bacterial species, or may be transformed using other technologies (e.g., biolistic transformation, WHISKER-mediated transformation, or other transformation methods) for the transfer of the DNA into the plant cell.

### Callus Cultures

Plant tissue cultures may advantageously be cultivated with a suitable bacterial species including, for example, Agrobacterium tumefaciens or Agrobacterium rhizogenes, for the transfer of the DNA into the plant cell, and are generally initiated from sterile pieces of a whole plant that may consist of pieces of organs, such as leaves or roots, or from specific cell types, such as pollen or endosperm. Many features of the explant are known to affect the efficiency of culture initiation, and the efficiency of culture initiation may also be affected by the composition of tissue culture medium, light intensity, temperature, humidity, or other environmental conditions. It is thought that any plant tissue can be used as an explant, if the correct conditions are found. Generally, younger, more rapidly growing tissue (or tissue at an early stage of development) is most effective for callus initiation. Explants cultured on the appropriate medium can give rise to an unorganized, growing, and dividing mass of cells (callus). In culture, callus can be maintained more or less indefinitely, provided that it is subcultured on to fresh medium periodically. During callus formation, there is some degree of de-differentiation, both in morphology (a callus is usually composed of unspecialized parenchyma cells) and metabolism.

Callus cultures are extremely important in plant biotechnology. Manipulation of the plant hormone ratios in the culture medium can lead to the development of shoots, roots, or somatic embryos from which whole plants can subsequently be produced (regeneration). Callus cultures can also be used to initiate cell suspension cultures that may be used to study plant transformation, gene regulation, and other aspects of plant growth and development.

### Cell Suspension Cultures

Callus cultures can typically be classified into one of two categories: compact or friable. In compact callus, the cells are densely aggregated, while in friable callus, the cells are only loosely associated with each other and the callus becomes soft and breaks apart easily. Friable callus provides the inoculum to develop cell-suspension cultures. Explants from some plant species or particular cell types tend not to form friable callus, particularly when cultured under conditions that do not promote the production of friable callus, making it difficult to initiate cell suspension cultures. The friability of the callus can sometimes be improved by manipulating the medium components, by repeated subculturing, and/or by culturing it on semi-solid medium (medium with a low concentration of gelling agent). When friable callus is placed into a liquid medium and agitated, single cells and/or small clumps of cells are released into the medium. Under certain conditions, these released cells continue to grow and divide, eventually producing a cell-suspension culture. Cell suspensions can be maintained relatively simply as batch cultures in conical flasks and can be propagated by repeated subculturing into fresh liquid tissue culture medium. After subculture, the cells continue to divide and the biomass of the culture increases as a result. Cell suspension cultures may advantageously be cultivated with, for example, Agrobacterium tumefaciens, Agrobacterium rhizogenes, or other suitable bacterial species capable of transferring DNA into the plant cell, or may be transformed using other suitable techniques.

### Shoot Tip and Meristem Culture

The tips of shoots (which contain the shoot apical meristem) can be cultured in vitro, producing clumps of shoots from either axillary or adventitious buds and may advantageously be cultivated with, for example, Agrobacterium tumefaciens, Agrobacterium rhizogenes, or other suitable bacterial species that may be used for the transfer of the DNA into the plant cell, or may be transformed using other suitable techniques known in the art. Shoot meristem cultures may be used for cereal regeneration; seedlings can be used as donor material.

### Embryo Culture

Embryos can be used as explants to generate callus cultures or somatic embryos. Immature or mature embryos may be used as explants for callus generation. Immature, embryo-derived embryogenic callus is a tissue often used in monocotyledon plant tissue culture regeneration and may advantageously be cultivated with, for example, Agrobacterium tumefaciens, Agrobacterium rhizogenes, or other suitable bacterial species that may be used for the transfer of the DNA into the plant cell, or may be transformed using other suitable techniques. Immature embryos are an intact tissue that is capable of cell division to give rise to callus cells that can differentiate to produce tissues and organs of a whole plant. Immature embryos can be obtained from the fertilized ears of a mature maize plant, for example, from plants pollinated using the methods of Neuffer et al. (1982, Growing maize for genetic purposes. In: Maize for Biological Research. W. F. Sheridan, Ed. UNIVERSITY PRESS, University of North Dakota, Grand Forks, N. Dak.). Exemplary methods for isolating immature embryos from maize are described by Green and Phillips (Crop Sci. 15:417-421 (1976)). Immature embryos are preferably isolated from the developing ear using aseptic methods and are held in sterile medium until use. The use of Agrobacterium in transformation of immature embryos is disclosed by Sidorov & Duncan, (2009, Methods in Molecular Biology: Transgenic Maize, vol. 526 Chapter 4, M. Paul Scott (Ed.)) and in U.S. Pat. No. 5,981,840.

### Microspore Culture

Haploid tissue can be cultured in vitro for example by using pollen or anthers as an explant and may advantageously be cultivated with, for example, Agrobacterium tumefaciens, Agrobacterium rhizogenes, or other suitable bacterial species that may be used for the transfer of the DNA into the plant cell, or may be transformed using other suitable techniques. Both callus and embryos can be produced from pollen. At least two approaches can be taken to produce cultures in vitro from haploid tissue. In the first, anthers (somatic tissue that surrounds and contains the pollen) are cultured on solid tissue culture medium. Pollen-derived embryos are subsequently produced via dehiscence of the mature anthers. The dehiscence of the anther depends both on its isolation at the correct stage and on the correct culture conditions. In some species, the reliance on natural dehiscence can be circumvented by cutting the wall of the anther. In the second method, anthers are cultured in liquid medium, and pollen released from the anthers can be induced to form embryos. Immature pollen can also be extracted from developing anthers and cultured directly.

Many of the cereals (rice, wheat, barley, and maize) require medium supplemented with plant growth regulators for pollen or another culture. Regeneration from microspore explants can be obtained by direct embryogenesis, or via a callus stage and subsequent embryogenesis.

Haploid tissue cultures can also be initiated from the female gametophyte (the ovule). In some cases, this may be a more efficient method than using pollen or anthers.

Plants obtained from haploid cultures may not be haploid as a result of chromosome doubling during the culture period. Chromosome doubling (which may be induced by treatment with, for example, chemicals such as colchicine) may be an advantage, as in many cases haploid plants are not the desired outcome of regeneration from haploid tissues. Such plants are often referred to as di-haploids, because they contain two copies of the same haploid genome.

Following transformation, whole plants may be regenerated from the transformed plant material following placement in suitable growth conditions and culture medium. The regeneration medium may contain antibiotics and/or herbicides, as appropriate, for selection of the transformed plant cells, depending on the presence of selectable marker genes that impart resistance or tolerance to such selective agents (i.e., antibiotics and/or herbicides). The plants so obtained can then be tested for the presence of the inserted DNA.

Whole Seed Transformation

Disclosed herein methods for transforming plant embryos within their seeds without cutting into the seed or otherwise substantially disrupting the tissues of the seed or the embryo. Standard methods known in the art require the creation of an explant from the seed by cutting through or removing the seed coat so that cells of the embryo can be directly contacted with a solution comprising an infecting bacterium that delivers a desired molecule into the plant cells (e.g., Agrobacterium—mediated transformation). The effectiveness of those other methods depends largely on the type of explant used and whether tissues of the cotyledon, leaf, hypocotyl, root, or some other organ of the seed or embryo is removed or disturbed, often by the fingers or a hand-held tool like a scalpel or forceps. The invasive nature of these methods requires special skills, tools, media, extensive tissue culture facilities and expertise, and many months to reliably recover healthy transgenic plants, especially on a high-throughput, commercial or industrial scale.

On the other hand, the teachings disclosed herein describe how embryos can be transformed without removing seed tissues or without physically disturbing tissues of the seed. In certain embodiments, “whole seeds”, i.e. seeds still connected to their seed coat and other tissues, can be transformed by exposing the whole seeds to a surfactant before a transformation step, e.g. before exposure to a transforming bacteria like Agrobacterium, or before biolistic procedures. The methods disclosed herein, including replacing the types of explants currently used in the art with whole seeds, circumvents several critical limiting factors in plant transformation and accelerates the recovery of transformants. This is due, in part, to the fact that the new methods do not require the disturbance or separation of tissues within or on the seed and can even permit the transformation of an embryo without removing or cutting through the seed coat. The whole seeds can remain substantially intact throughout the transformation process, allowing the embryos to remain safe within the seed and the natural protections the seed affords. The transformed embryos are also able to continuously access nutrients within the seed as they naturally do even during the transformation process and subsequent selection and recovery steps. Accordingly, whole seeds are provided that have been exposed to the surfactant compositions disclosed herein and are ready for transformation. Moreover, transformed whole seeds are provided that have little to no disturbance of the seed coat.

The present methods do not require separation of cotyledons, wounding of the embryo, or trimming of the root and this improves post-transformation root formation and co-culture and/or post-transformation recovery. The recovery and transformed plants is more akin to germination under the current methods than the additional steps of callus establishment and/or artificially induced organogenesis that other methods of the art require. The consequences of using the techniques taught herein can surprisingly reduce the time needed to recover transformed plants from the approximately 3-12 months commonly required by methods currently disclosed in the art down to approximately 2-3 months.

Cell transformation (including plant cell transformation) may involve the construction of an expression vector which will function in a particular cell. Such a vector may comprise DNA that includes a gene under control of, or operatively linked to, a regulatory element (for example, a promoter) that is operable in a plant cell. The expression vector may contain one or more such operably-linked gene/regulatory element combinations. The vector(s) may be in the form of at least one plasmid and can be used alone or in combination with other plasmids to provide transformed cells using transformation methods as described herein to incorporate transgene(s) into the genetic material of a plant cell.

Plant cell expression vectors may include at least one genetic marker (alternately referred to as a “selectable marker gene”), operably linked to a regulatory element (a promoter, for example) that allows transformed cells containing the marker to be recovered either by negative selection (i.e., inhibiting growth of cells that do not contain the selectable marker gene) or by positive selection (i.e., screening for the product encoded by the genetic marker). Many selectable marker genes suitable for plant transformation are well known in the art and include, for example, genes that encode enzymes that metabolically detoxify a selective chemical agent such as, for example, an antibiotic or an herbicide, or genes that encode an altered target which may be insensitive to the inhibitor. Positive selection methods are also known in the art. The individually employed selectable marker gene may accordingly permit the selection of transformed cells while the growth of cells that do not contain the inserted DNA can be suppressed by the selective compound. Different selectable marker gene(s) and selection methods may be employed for the transformation of different plant species, different tissues, or for the purposes of modifying plant transformation efficiencies, for example. Examples of suitable selectable markers include, but are not limited to, resistance or tolerance to Kanamycin, G418, Hygromycin, Bleomycin, Methotrexate, Phosphinothricin (Bialaphos), Glyphosate, Imidazolinones, Sulfonylureas and Triazolopyrimidine herbicides, such as Chlorosulfuron, Bromoxynil, and DALAPON.

In addition to a selectable marker, it may be desirable in some embodiments to use a reporter gene. In some embodiments a reporter gene may be used without a selectable marker (i.e., through visual selection alone by inspection for presence of the reporter gene-encoded product rather than through the use of a positive or negative selection technique). Reporter genes are genes which typically do not provide a growth advantage to the recipient organism or tissue. The reporter gene typically encodes for a protein which provides for some phenotypic change or enzymatic property. Some commonly used suitable reporter genes include, but are not limited to, those that encode beta-glucuronidase (GUS), luciferase, or fluorescent proteins such as green fluorescent protein (GFP), yellow fluorescent protein (YFP, essentially as disclosed in U.S. Pat. No. 7,951,923), or other fluorescent proteins.

Typically, following the introduction of the gene(s) to expressed in the plant cell, three phases may be observed. In the first phase, the inserted gene(s) are transiently expressed, though they may not be stably inserted into the genome of the recipient cell. This transient expression may result from expression of the introduced DNA, though integration of the DNA into the recipient cell genome may not have yet occurred. In some embodiments, this first phase may last for up to 24 hours, up to 48 hours, up to 72 hours, up to 96 hours, or up to one week following transformation.

A second phase may be observed on tissue culture medium during which stable sectors of transformed plant cells are formed. These stable sectors comprise dividing cells in which the introduced gene(s) have been stably inserted into the genome. Expression of the introduced gene(s) continues after the introduced DNA has been cleared as a result of the normal replication of the cellular DNA. The stable sectors will continue to divide and grow and may produce shoots. In some embodiments, shoot production may be stimulated for example by the addition of suitable chemicals such as plant hormones. Following shoot production, a third phase begins during which stably transformed plants are regenerated from transformed plant cells. Regenerated plants may be grown on suitable tissue culture medium and may produce roots, leaves, and other organs. Typically, regenerated plants are transferred to soil for continued cultivation in, for example, a greenhouse or other suitable environment.

Regardless of the transformation technique utilized, the gene(s) to be inserted into the genome of the recipient plant cell, and/or to be expressed in the recipient plant cell, can be incorporated into a gene transfer vector adapted to express the foreign gene in the plant cell by including in the vector a promoter that is operable in a plant cell. In addition to plant promoters, promoters from a variety of sources can be used efficiently in plant cells to express foreign genes. For example, promoters of bacterial origin, such as the octopine synthase promoter, the nopaline synthase promoter, the mannopine synthase promoter; promoters of viral origin, such as the 35S and 19S promoters of cauliflower mosaic virus (CaMV), a promoter from sugarcane bacilliform virus, and the like may be used. Plant-derived promoters include, but are not limited to ribulose-1,6-bisphosphate (RUBP) carboxylase small subunit (ssu), beta-conglycinin promoter, phaseolin promoter, ADH (alcohol dehydrogenase) promoter, heat-shock promoters, ADF (actin depolymerization factor) promoter, and tissue specific promoters.

Promoters may also contain certain enhancer sequence elements that may improve the transcription efficiency. Typical enhancers include, but are not limited to, alcohol dehydrogenase 1 (ADH1) intron 1 and ADH1-intron 6. Constitutive promoters, which direct continuous gene expression in nearly all cells types and at nearly all times (e.g. actin, ubiquitin, CaMV 35S), may also be used. Tissue specific promoters are responsible for gene expression in specific cell or tissue types, such as the leaves or seeds. Examples of other promoters that may be used include those that are active during a certain stage of the plant's development, as well as active in specific plant tissues and organs. Examples of such promoters include, but are not limited to, promoters that are root specific, pollen-specific, embryo specific, corn silk specific, cotton fiber specific, seed endosperm specific, and phloem specific.

Under certain circumstances, it may be desirable to use an inducible promoter. An inducible promoter is responsible for expression of genes in response to a specific signal, such as physical stimulus (e.g. heat shock genes); light (e.g. Ribulose-bis-phosphate 1,5 carboxylase); hormone (e.g. glucocorticoid) accumulation; antibiotic (e.g. Tetracycline); metabolites; and stress (e.g. drought). Other desirable transcription and translation elements that function in plants also may be used, such as, for example, 5′ untranslated leader sequences, and 3′ RNA transcription termination and poly-adenylate addition signal sequences. Any suitable plant-specific gene transfer vector may be used.

Transgenic crops containing insect resistance (IR) traits are prevalent in commercially grown crop plant species, as are crops containing herbicide tolerance (HT) traits. Commercial transgenic crops combining IR and herbicide tolerance (HT) traits are also widely grown. These include combinations of IR traits conferred by Bacillus thuringiensis (B.t.) insecticidal proteins and HT traits such as tolerance to Acetolactate Synthase (ALS) inhibitors such as Sulfonylureas, Imidazolinones, Triazolopyrimidine, Sulfonanilides, and the like, Glutamine Synthetase (GS) inhibitors such as Bialaphos, Glufosinate, and the like, 4-HydroxyPhenylPyruvate Dioxygenase (HPPD) inhibitors such as Mesotrione, Isoxaflutole, and the like, 5-EnolPyruvylShikimate-3-Phosphate Synthase (EPSPS) inhibitors such as Glyphosate and the like, and Acetyl-Coenzyme A Carboxylase (ACCase) inhibitors such as Haloxyfop, Quizalofop, Diclofop, and the like. Other examples are known in which transgenically provided proteins provide plant tolerance to herbicide chemical classes such as phenoxy acids herbicides and pyridyloxyacetates auxin herbicides (see WO 2007/053482 A2), or phenoxy acids herbicides and aryloxyphenoxypropionates herbicides (see WO 2005/107437A1). The ability to control multiple pest problems through IR traits is a valuable commercial product concept, and the convenience of this product concept is enhanced if insect control traits and weed control traits are combined in the same plant. Further, improved value may be obtained via single plant combinations of IR traits conferred by a B.t. insecticidal protein with one or more additional HT traits such as those mentioned above, plus one or more additional input traits (e.g. other insect resistance conferred by B.t.-derived or other insecticidal proteins, insect resistance conferred by mechanisms such as RNAi and the like, disease resistance, stress tolerance, improved nitrogen utilization, and the like), or output traits (e.g. high oils content, healthy oil composition, nutritional improvement, and the like). Such combinations may be obtained through, e.g., conventional breeding (e.g. a breeding stack), and/or jointly as a novel transformation event involving the simultaneous introduction of multiple genes (e.g. a molecular stack), and/or through genome editing methods that allow for the insertion of genes at a pre-determined location in the genome of the target cell or organism. Benefits include the ability to manage insect pests and improved weed control in a crop plant that provides secondary benefits to the producer and/or the consumer.

Genetic modification of crop plants may also be used to provide additional benefits to the plant. Such benefits may include, without limitation, modified flavor profiles, modified amino acid content and/or quality, modified total protein content and/or quality, modified oil content and/or quality, altered color, improved resistance to abiotic stresses such as heat, drought, cold, and/or flooding, improved post-harvest shelf stability, improved digestibility, and/or other desirable traits.

The methods described herein are broadly applicable to a variety of plant species and varieties including monocotyledons and dicotyledons. Crops of interest include but are not limited to corn (Zea mays), Brassica sp. (e.g., B. napus, B. rapa, B. juncea), particularly those Brassica species useful as sources of seed oil, alfalfa (Medicago sativa), rice (Oryza sativa), rye (Secale cereale), sorghum (Sorghum bicolor, Sorghum vulgare), pea (Pisum sativum), millet (e.g., pearl millet (Pennisetum glaucum), proso millet (Panicum miliaceum), foxtail millet (Setaria italica), finger millet (Eleusine coracana)), sunflower (Helianthus annuus), quinoa (Chenopodium quinoa), chicory (Cichorium intybus), lettuce (Lactuca sativa), safflower (Carthamus tinctorius), wheat (Triticum aestivum), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatus), cassava (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oil palm (Elaeis guineensis), poplar (Populus spp.), eucalyptus (Eucalyptus spp.), oats (Avena sativa), barley (Hordeum vulgare), vegetables, ornamentals, and conifers. The methods herein can be used with cells at various stages of development, e.g., immature embryos. Thus, the methods described herein can be used to transform maize immature embryos. The size of immature embryos used in conjunction with the methods described herein can vary. For example, immature embryos can be greater than or equal to 1.5 mm and less than or equal to 2.5 mm in length.

The external environment the plant cells are maintained in after transformation according to the methods described herein can be controlled. For example, temperature, pH, and the components in the growth medium (e.g., salts and/or plant hormones) the cells are exposed to after transformation according to the methods described herein are varied. One of those variables is the amount of light the cells are exposed to. The methods described herein can include exposing the plant cells to common 18 hour light/6 hour dark protocols or alternatively to continuous light after transformation. For example, cells treated according to the methods described herein can be exposed to 24-hour white fluorescent light conditions for weeks after treatment, e.g., until the regeneration and/or plantlet isolation stages of plant preparation.

An additional method includes preparing a liquid medium containing a surfactant, exposing plant cells to the surfactant-containing medium, and then removing the surfactant-containing medium prior to transformation. The surfactant-treated plant cells are referred to as “pre-conditioned” cells and are more amenable to transformation than cells that are not pre-conditioned.

As used herein, a “non-conditioned” or “unconditioned” plant cell or plant cells have not been conditioned by exposure to a surfactant. For example, “non-conditioned” or “unconditioned” plant cells have not been exposed to a surfactant for more than 1 second (sec), 5 sec, 10 sec, 15 sec, 30 sec, 1 min, 2 min, 5 min, 10 min, 15 min, 30 min, 45 min, 1 hr, 1.5 hr, 2 hr, 3 hr, 4 hr, 5 hr, 6 hr, 8 hr, 10 hr, 12 hr, or 1-60 sec, 1-90 sec, 1 sec to 5 min, 1 sec to 10 min, 1 sec to 15 min, 1 sec to 30 min, 1 sec to 45 min, 1 sec to 1 hr, 1 sec to 1.5 hr, 1 sec to 2 hr, or 1 sec to 3 hr. In specific embodiments, non-conditioned plant cells can be infected by a bacterium or virus comprising a polynucleotide in a medium that does not contain an effective amount of surfactant and subsequently exposed to an effective amount of a surfactant, such as during a co-culture step or after a co-culture step wherein the bacterium or virus has been removed from the plant cell prior to the addition of the effective amount of surfactant.

In certain embodiments, plant cells are not pre-conditioned but instead exposed to surfactant only after infection, for example, only after exposing the plant cells to the transforming bacteria and/or after the plant cells have been separated from the bacteria that failed to adhere to the plant surfaces during infection. In certain embodiments, plant cells are exposed to surfactants only during the coculturing step. In certain embodiments, the plant cells are exposed to surfactant only at even later steps in the transformation process, for example, during the various steps of transgenic selection and recovery, including callus induction, shoot elongation, regeneration, and/or rooting, etc. In specific embodiment, plant cells can be exposed to surfactants at multiple steps during the transformation process. Specific surfactants can be selected for use in transformation based on the stage of transformation at which the individual surfactant is exposed to the plant cell.

In certain embodiments, plant cells are exposed to surfactant before infection (e.g. during a first step) in the transformation process, for example, during the seed imbibition step and/or before explants are prepared. In certain embodiments, improved transformation efficiencies can be achieved at other times prior to infection, e.g. during and/or after explant preparation.

Protocols and methods for transforming plants include, for example and without limitation, transformation by Agrobacterium species (e.g., A. tumefaciens or A. rhizogenes) or other suitable bacterial species (e.g., Ensifer species or Ochrobactrum species), or transformation by biolistic methods or transformation by other methods. Any method useful for plant transformation can be employed in conjunction with the methods described herein. The examples below provide embodiments of methods demonstrating the effectiveness of the methods described herein, but are not intended to be limitations on the scope of the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

### Embodiments of the Invention Include

1. A method of transforming a plant cell with a polynucleotide by exposing the plant cell to a surfactant or surfactant containing medium before the plant cell is infected by a bacterium containing the polynucleotide, or a virus containing the polynucleotide, wherein the bacterium or virus is capable of transforming the plant cell by infecting the plant cell. In particular aspects of embodiment 1, an effective amount of surfactant is exposed to the plant cell.
2. The method of embodiment 1 wherein exposing the plant cell to a surfactant comprises exposing a seed comprising the plant cell to surfactant.
3. The method of embodiment 2 further comprising removing the plant cell from surfactant before exposing the plant cell to the bacterium or virus.
4. The method of embodiment 2 further comprising exposing the plant cell to an additional treatment of surfactant after the plant cell has been infected by the bacterium or virus.
5. The method of embodiment 4 wherein the additional treatment of surfactant is applied at least 1 hour (e.g., 1-24 hr, 1-12 hr, 1-6 hr, 1-4 hr, 1-3 hr, 1-2 hr, 1-1.5 hr) after the plant cell has been exposed to the bacterium or virus (or after said bacterium or virus have been removed).
6. The method of embodiment 2 wherein the surfactant is applied during a co-culture step.
7. The method of embodiment 2 wherein the surfactant is applied during a step selected from the group comprising tissue culture steps to promote transgenic event recovery, including but not limited to selection, callus induction, shoot elongation, regeneration, and rooting.
8. The method of embodiment 2 wherein the seed imbibes the solution for at least 1 hour, at least 2 hours, at least 3 hours, at least 4 hours or at least 1-2 hrs, 1-3 hours, 1-4 hours, 1-6 hours, 2-4 hours, 2-6 hours, 3-6 hours, 4-6 hours, 1-24 hours, 1-12 hours, 4-24 hours, or 4-48 hours.
9. A method of transforming a non-conditioned plant cell with a polynucleotide by exposing the non-conditioned plant cell to a surfactant after the non-conditioned plant cell is infected by a bacterium containing the polynucleotide, or a virus containing the polynucleotide, wherein the bacterium or virus is capable of transforming the plant cell by infecting the plant cell. In particular embodiments of embodiment 9, an effective amount of surfactant is exposed to the plant cell.
10. The method of embodiment 9 wherein exposure of the plant cell to surfactant occurs during a co-culture step.
11. The method of embodiment 9 wherein exposure of the plant cell to surfactant occurs after a co-culture step.
12. The method of any one of embodiments 1-11 wherein the bacterium is a species selected from the group comprising Agrobacterium, Ochrobactrum, Rhizobium, Ensifer and the collective bacterial strains termed Transbacter™
13. A method of transforming a plant cell with a nucleotide sequence by a physical or chemical (i.e. biolistic) transformation procedure that comprises exposing the plant cell to a surfactant and wherein the biolistic transformation procedure comprises a bombardment step wherein the plant cell is bombarded by a particle capable of transforming the plant cell with the nucleotide sequence. In particular aspects of embodiment 13, an effective amount of surfactant is exposed to the plant cell.
14. The method of embodiment 13 wherein the plant cell is exposed to a surfactant before the bombardment step.
15. The method of embodiment 13, wherein the plant cell is exposed to a surfactant during or after the bombardment step.
16. The method of embodiment 13 wherein the plant cell is exposed to a surfactant during at least two steps of the biolistic transformation process.
17. A method of transforming a plant cell with a macromolecule by a biolistic transformation procedure that comprises exposing the plant cell to a surfactant and wherein the biolistic transformation procedure comprises a bombardment step wherein the plant cell is bombarded by a particle capable of transforming the plant cell with the nucleotide sequence. In particular aspects of embodiment 17, an effective amount of surfactant is exposed to the plant cell.
18. The method of embodiment 17 wherein the plant cell is exposed to a surfactant before the bombardment step.
19. The method of embodiment 17 wherein the plant cell is exposed to a surfactant during or after the bombardment step.
20. A method of transforming a plant cell by an electroporation treatment capable of delivering a polynucleotide into a plant and wherein the plant cell is exposed to a surfactant. In particular aspects of embodiment 20, an effective amount of surfactant is exposed to the plant cell.
21. The method of embodiment 20 wherein the plant cell is subjected to the electroporation treatment during or after it is exposed to a surfactant.
22. The method of embodiment 21 wherein the plant cell is exposed to a surfactant before the electroporation treatment.
23. A method of transforming a plant cell with a macromolecule by an electroporation treatment capable of delivering the macromolecule into a plant and wherein the plant cell is exposed to a surfactant. In particular aspects of embodiment 23, an effective amount of surfactant is exposed to the plant cell.
24. The method of embodiment 23 wherein the plant cell is exposed to a surfactant before the electroporation treatment.
25. The method of embodiment 23 wherein the plant cell is exposed to the surfactant during or after the electroporation treatment.
26. The methods of any of the embodiments 1-25 wherein the surfactant is in a solution at a concentration from about 0.0001% to about 0.1%.
27. The method of embodiments 17 or 23 wherein the macromolecule is a polypeptide.
28. The method of embodiment 27 wherein the macromolecule is a peptide comprising at least one CRISPR nuclease.
29. The method of embodiments 17 or 23 wherein the macromolecule is a ribonucleic protein.
30. The method of embodiment 27 wherein the polypeptide is capable of creating mutations in a nucleotide sequence of the plant cell.
31. The method of embodiment 27 or 30 wherein the polypeptide comprises at least one CRISPR nuclease.
32. A method of improving transformation of plant cells comprising:
   i) pre-conditioning plant cells by exposure to a surfactant containing medium,
   ii) removing said plant cells from said surfactant containing medium, and
   iii) introducing at least one polynucleotide sequence into said plant cells. In particular aspects of embodiment 32, an effective amount of surfactant is exposed to the plant cell.
33. The method of any one of embodiments 1-32, wherein said surfactant containing medium comprises a non-ionic surfactant.
34. The method of any one of embodiments 1-32, wherein said surfactant containing medium comprises a surfactant selected from the group consisting of Break-Thru S233, Break-Thru S240, Break-Thru S279, Break-Thru S301, and Pluronic F-68.
35. The method of embodiment 32, wherein said surfactant containing medium comprises surfactant at a concentration of 0.001-0.1% (v/v).
36. The method of any one of embodiments 1-35 wherein said exposure to a surfactant containing medium lasts for 5-60 minutes.
37. The method of embodiment 32 wherein said introducing one or more polynucleotide sequence(s) includes the use of Agrobacterium cells harboring a plant transformation construct.
38. The method of embodiment 37 wherein said Agrobacterium cells harboring a plant transformation construct comprise a binary vector.
39. The method of embodiment 37 wherein said Agrobacterium cells harboring a plant transformation construct comprise a superbinary vector.
40. The method of embodiment 32 wherein said improving transformation of plant cells comprises an increased percentage of plant cells exhibiting transient expression of said at least one polynucleotide sequence relative to control plant cells not exposed to said surfactant containing medium.
41. The method of embodiment 32 wherein said improving transformation of plant cells comprises an increased percentage of callus pieces developing stably transformed sectors.
42. The method of embodiment 32 wherein said improving transformation of plant cells comprises an increased number of transformed plants regenerated from transformed tissue.
43. The method of embodiment 32 wherein said plant cells are derived from a monocot.
44. The method of embodiment 43 wherein said plant cells are derived from Zea mays, Oryza sativa, Setaria viridis, Sorghum bicolor, Triticum aestivum, or Saccharum sp.
45. The method of embodiment 32 wherein said plant cells are derived from a dicot.
46. The method of embodiment 45 wherein said plant cells are derived from Pisum sativum, Lactuca sativa, or Solanum lycopersicum.
47. The method of embodiment 32 wherein said at least one polynucleotide sequence comprises a polynucleotide sequence that shares at least 80% sequence identity with a sequence selected from the group of sequences consisting of SEQ ID NOs:1 and 15, or that encodes a protein that shares at least 80% sequence identity with a sequence selected from the group of sequences consisting of SEQ ID NOs:2 and 16.
48. The method of embodiment 32 wherein said at least one polynucleotide sequence comprises a polynucleotide sequence that shares at least 80% sequence identity with a sequence selected from the group of sequences consisting of SEQ ID NOs:3, 5, and 7, or that encodes a protein that shares at least 80% sequence identity with a sequence selected from the group of sequences consisting of SEQ ID NO:4, 6, and 8.
49. The method of embodiment 32 wherein said introducing at least one polynucleotide sequence comprises biolistic transformation.
50. The method of embodiment 32 wherein said at least one polynucleotide sequence encodes at least one CRISPR nuclease.
51. The method of embodiment 50 wherein said pre-conditioning results in improved genome editing relative to control cells not exposed to said surfactant containing medium.
52. A method of transforming a seed with a polynucleotide or polypeptide by
   i) exposing the seed comprising a seed coat to a surfactant, and
   ii) exposing the seed to a bacterium containing the polynucleotide or polypeptide, or a virus containing the polynucleotide or polypeptide, wherein the bacterium or virus is capable of transforming the plant cell by infecting the plant cell.
53. The method of embodiment 52 wherein the seed is a whole seed.
54. The method of embodiment 52 or 53 wherein the seed coat remains attached to the seed.
55. The method of embodiment 52 or 53 wherein the seed coat remains substantially intact.
56. The method of any one of embodiments 52-55 wherein the seed has been subjected to sonication.
57. A seed containing a transformed embryo wherein the seed has been exposed to a surfactant. The following examples are offered by way of illustration and not by way of limitation.

### EXPERIMENTAL

### Example 1—Maize Transformation Materials and Embryo Isolation

Plant Materials and Embryo Isolation

Zea mays (B 104) plants were grown in the greenhouse tinder 50% metal halide, 50% high pressure sodium lights (4b daylength, 28° C. day/22° C. night, 40-50% minimum relative humidity). The plants were self-pollinated and produced ears suitable for embryo transformation. Ten to thirteen days after pollination, ears were harvested and surface-sterilized in a 20% (v/v) solution of household bleach containing 005% (v/v) Tween 20 for 20 minutes while stirring Following bleach sterilization, the ears were rinsed in sterile water 3-5 times for 5 min/each rinse. Immature zygotic embryos (1.82-2.2 mm) were aseptically isolated from each ear and randomly distributed into micro-centrifuge tubes containing liquid infection media (MS salts, 4.33 gm/L; MS modified Vitamin Solution (Phytotechnology M557) [1000×], 1.00 mL/L; L-proline, 700.0 mg/L; sucrose, 68.5 gm/L: glucose, 36.0 gm/L; 2,4-D, 1.50 mg/L) and the pH was adjusted to 5.2.

Agrobacterium Culture Initiation

Glycerol stocks of Agrobaterium containing the appropriate vectors were stored at −80° C. until ready to use. A loop from the frozen glycerol was streaked on AB minimal medium plates containing appropriate antibiotics for plasmid maintenance and plates were grown at 20-25° C. for 3 days in the dark. A single colony was then picked and streaked onto YEP1 plate containing the same antibiotics and was incubated at 28° C. for 1-3 days.

Agrobacterium Culture

On the day of the experiment, Agrobacterium colonies were taken from the YEP plate, suspended in 10-15 ml, of infection medium in a 50 mL disposable tube, and the cell density adjusted to OD600=0.2-0.4 nm for AGL1 Agrobacterium harboring binary vector 131440 (SEQ ID NO:9) and 0,8-1.1 for LBA4404 Agrobacterium harboring superbinary vector 130571 (SEQ ID NO: 10) Agrobacterium cultures were then placed on a rotary shaker at 120-130 rpm, room temperature, while embryos dissection was performed.

Preconditioning Treatment

For the preconditioning treatment, 0.1-0.5% (v/v) of surfactant was added to infection media. Immature zygotic embryos between 1.8-2.2 mm in size were isolated and pooled from the sterilized maize kernels and placed either in 1.75 ml of the infection medium alone or in infection media comprising the appropriate surfactant (“preconditioning medium”). The preconditioning treatment lasted between 5 min-60 min and was performed at room temperature. After all embryos were isolated and preconditioned, the preconditioning media was removed by pipetting from the embryos and discarded.

Agrobacterium Infection and Co-Cultivation

Following pre-conditioning, 1.75 ml of Agrobacterium suspension diluted to the appropriate OD600 concentration was added to each tube. Tubes were then inverted to mix and placed on rocker shaker for 10-15 min at room temperature. Infected embryos were transferred onto co-cultivation media (MS salts, 4.33 gm/L; MS modified Vitamin Solution (Phytotechnology M557) [1000×], 1.00 mL/L: L-proline, 700.0 mg/L; casein enzymatic hydrolysate 100.0 mg/L; Dicamba 3.0 mg/l.; sucrose, 300 gm/l; Gelzan™,2,00 gm/L/; AgNo₃, 15.0 mg/L.; Acetosyringone, 200 μM), and pH adjusted to 5.6. Infected embryos were oriented with the scutellum facing up, and incubated for 3-5 days in 24 hr light (50 μmol m²s⁻¹) at 25° C.

Callus Selection and Regeneration of Putative Events

Following the co-cultivation period, embryos were transferred to resting media (MS salts, 4.33 gm/L;MS modified Vitamin Solution (Phytotechnology M557) [1000×], 1.00 mL/L; L-proline, 700.0 mg/L; MES, 500.0 mg/L; casein enzymatic hydrolysate 100.0 m/L; Dicamba, 3.0 mg/L; sucrose, 30.0 gm/L; Gelzan™2.0 gm/L; AgNo₃, 15.0 mg/L; Cefotaxime, 250.0 mg/L) without selective agent and incubated in the light for 7-10 days at 27° C. Embryos were then transferred onto Selection 1 media (MS salts, 4.33 gm/L; MS modified Vitamin Solution (Phytotechnology M557) [1000×], 1.00 mL/L; L-proline, 700.0 mg/L; MES 500.0 mg/I.; casein enzymatic hydrolysate 100.0 mg/L; Dicamba, 3.0 mg/L; sucrose, 30.0 gm/L; Gelzan™ 2.0 gm/L; AgNO₃, 15.0 mg/L; Cefotaxime, 250.0 mg/L) containing 3-5 mg/I. bialaphos or 100-130 mg/l paromomycin with pH adjusted to 5.8. Plates were incubated tinder 24 hours light with light intensity of 50 μmol m²s⁻¹ for 7-14 days at 27° C.

Embryos with proliferating embryogenic calli were then transferred onto Selection 2 media (MS salts, 4.33 gm/L MS modified Vitamin Solution (Phytotechnology M557) [1000×], 1.00 mL/L; L-proline, 700.0 mg/L; M-ES 500.0 mg/L; casein enzymic hydrolysate 100.0 mg/L; Dicamba, 3.0 mg/L: sucrose, 30.0 gm/L; Gelzan™ 2.0 gm/L AgNo₃, 15.0 mg/L; Cefotaxime, 250.0 mg/L) and containing 3-5 mg/L bialaphos or 100-130 mg/l paromomycin with pH adjusted to 5.8. Plates were incubated under 24 hours light with light intensity of 50 μmol m²s⁻¹ for 14 days at 27° C. This selection step allows transgenic callus to further proliferate and differentiate. The callus selection period lasted three to four weeks. Embryogenic callus was transferred onto Regeneration 1 media (MIS salts, 4.33 gm/L; MS modified Vitamin Solution (Phytotechnology M557) [I000×], 1.00 mL/L; L-proline, 350.0 mg/L: MES, 250.0 mg/L; casein enzymatic hydrolysate 50.0 mg/L; NAA 0.500 mg/L; ABA 2.00 mg/L; BA 1.50 mg/l.; sucrose, 45.0 gm/L; Gelzan™, 2.0 gm/; AgNo₃ 1.00 mg/L: Cefotaxime, 125.0 mg/L) and containing 3-5 mg/L bialaphos or 100-130 mg/I paromomycin with pH adjusted to 5.7, and cultured for 4-7 days at 27° C. under the same light regime. Calli with shoot buds were transferred onto Regeneration 2 media in phytatrays (MS salts, 4.33 gm/L; MS modified Vitamin Solution (Phytotechnology M557) [1000×], 1.00 mL/L; sucrose, 60.0 gm/i; Gelzan™ 2.3 gm/L; Cefotaxime, 125.0 mg/L) and containing 3-5 mg/L bialaphos or 100-130 mg/l paromomycin. The cultures were incubated under 21 hours light with light intensity of 50 μmol m²s⁻¹ for 14-21 days at 27° C. Plantlets with roots were transferred to plant robusting media (MS salts, 4.33 gm/L (Sigma-Aldrich, St. Louis, Mo.); MS modified Vitamin Solution (Phytotechnology M557) [1000×], 1.00 mL/L; myo-Ilnositol 100 mg/L sucrose, 30.0 gm/L; Gelzan™, 200 gm/L; Cefotaxime, 10 (mg/L) and Gelzan™, 2.0 gm/L in phytatrays (Sigma-Aldrich, St. Louis, Mo.), with pH adjusted to 5.7. Cultures were incubated under 16/8 hours light 90-120 μmol m²s⁻¹/dark for 7 days at 27° C. DNA was isolated from 30-50 mg leaf tissue from transgenic plantlets for molecular analysis.

### Example 2—Methods for Quantifying GFP Expression

Transient GFP expression was observed in transformed tissues 2-5 days after co-cultivation with Agrobacterium. The tissues were observed under a stereomicroscope using NIGHTSEA Fluorescence Leica EZ4 Adapter which includes a Royal Blue light source (440-460 nm) and 2 filter sets for GFP (500 nm longpass or 500-560 nm green only bandpass).

GFP Transient Expression was Evaluated Using Two Methods, as Described Below.

1) PerkinElmer Plate Reader

Randomly selected tissues from different treatments were sampled and placed into a 96 well strip plate. Multiple replicates of each treatment were included in the same plate. The plate was inserted into EnSpire Multimode Plate Reader 2300 (PerkinElmer, Turku, Finland). The plate reader was designed for top fluorescence absorbance and scanning. The protocol was optimized for Monochromator absorbance cutoff 230. The excitation wavelength was set to 488 nm and emission wavelength to 510 nm. The measurement height was at 9.5 mm. The flash power was at 100% and number of flashes and flashes integrated were 100 (Manual for Multimode Detection, PerkinElmer). PerkinElmer EnSpire software converts GFP fluorescence absorbance readings to emission numbers, reported as relative fluorescence units or RFUs.

2) Relative GFP expression in all tissues

A visual scoring scale procedure was developed for rating GFP expression in each infected tissue after coculture with Agrobacterium. Tissues were scored on a scale from 0-3, with a score of 0 representing no apparent GFP expression and a score of 3 representing the strongest GFP expression. Plates containing transformed tissues were observed under stereomicroscope using a GFP filter as described above.

### Example 3—Transient GFP Expression in Maize Following Binary Vector Transformation

Transient expression of GFP was measured following co-cultivation of maize tissue with AGL1 Agrobacterium cells harboring binary vector 131440. The effect of pre-conditioning medium comprising 0.01% (v/v) Break-Thru S233 was tested. Background fluorescence levels were calculated from fluorescence measurements of eight untransformed immature embryos. Twenty-four immature embryos were selected at random from 1) the group of immature embryos that were pre-conditioned with Break-Thru S233 and 2) the group of immature embryos that were not pre-conditioned. Table 1 shows the results of quantification of this transient GFP expression.

TABLE 1
Transient GFP expression after 131440
co-cultivation in experiment ZM2

| | background | untreated | pre-conditioned |
|---|---|---|---|
| | 3207 | 4394 | 61207 |
| | 2834 | 3842 | 58912 |
| | 1712 | 2187 | 49496 |
| | 1062 | 1913 | 30588 |
| | 543 | 1866 | 28044 |
| | 513 | 1713 | 22197 |
| | 314 | 1398 | 20774 |
| | 305 | 1366 | 19936 |
| | | 990 | 19891 |
| | | 987 | 19075 |
| | | 910 | 19013 |
| | | 748 | 12197 |
| | | 667 | 9467 |
| | | 662 | 8689 |
| | | 516 | 8617 |
| | | 485 | 6445 |
| | | 483 | 4390 |
| | | 467 | 2298 |
| | | 428 | 1508 |
| | | 347 | 1109 |
| | | 294 | 585 |
| | | 283 | 576 |
| | | 219 | 552 |
| | | 187 | 469 |
| Average | 1311.2 ± | 1139.6 ± | 16918.1 ± |
| | 1156.9 | 1089.5 | 17962.7 |
| Avg. + 3XStDev | 4782.1 | | |

Immature embryos were scored as positive for GFP expression if a fluorescence value higher than three standard deviations above background values was observed.

Table 1 shows that none of the 24 untreated embryos were scored as positive, while 16 of the 24 pre-conditioned embryos were scored as positive for transient GFP expression. While the standard deviations were quite large for the fluorescence values, this is an expected result because both GFP-expressing and non-expressing immature embryos were assessed as a result of the random choice of embryos for testing. These results show a substantial improvement in transient GFP expression in maize immature embryos resulting from the Break-Thru S233 pre-conditioning treatment following co-cultivation with Agrobacterium harboring a binary vector.

### Example 4—Transient GFP Expression in Maize Following Superbinary Vector Transformation

Transient expression of GFP was measured following co-cultivation of maize tissue with LBA4404 Agrobacterium cells harboring superbinary vector 130571. The effect of pre-conditioning medium comprising 0.01% (v/v) Break-Thru S233 was tested. Background fluorescence levels were calculated from fluorescence measurements of eight untransformed immature embryos. Twenty-four immature embryos were selected at random from 1) the group of immature embryos that were pre-conditioned with Break-Thru S233 and 2) the group of immature embryos that were not pre-conditioned. Table 2 shows the results of quantification of this transient GFP expression.

TABLE 2
Transient GFP expression after 130571
co-cultivation in experiment ZM1

| | background | untreated | pre-conditioned |
|---|---|---|---|
| | 2935 | 6814 | 129592 |
| | 1439 | 2160 | 77719 |
| | 1005 | 1924 | 30006 |
| | 950 | 1230 | 10001 |
| | 359 | 914 | 7261 |
| | 178 | 781 | 6527 |
| | 142 | 570 | 5695 |
| | 98 | 441 | 5492 |
| | | 441 | 3559 |
| | | 435 | 3378 |
| | | 358 | 3165 |
| | | 335 | 2283 |
| | | 217 | 2057 |
| | | 199 | 1840 |
| | | 192 | 1108 |
| | | 182 | 662 |
| | | 178 | 355 |
| | | 174 | 280 |
| | | 167 | 259 |
| | | 166 | 242 |
| | | 160 | 184 |
| | | 141 | 182 |
| | | 132 | 159 |
| | | 91 | 126 |
| Average | 888.2 +/− | | |
| | 961.8 | | |
| Avg + 3XStDev | 3773.7 | | |

Immature embryos were scored as positive for GFP expression if a fluorescence value higher than three standard deviations above background values was observed.

Table 2 shows that one of the 24 untreated embryos were scored as positive, while 8 of the 24 pre-conditioned embryos were scored as positive for transient GFP expression. While the standard deviations were quite large for the fluorescence values, this is an expected result because both GFP-expressing and non-expressing immature embryos were assessed as a result of the random choice of embryos for testing. These results show a substantial improvement in transient GFP expression in maize immature embryos resulting from the Break-Thru S233 pre-conditioning treatment following co-cultivation with Agrobacterium harboring a superbinary vector.

GFP expression in maize immature embryos in experiment ZM1 were also scored according to the relative GFP expression protocol, scoring each embryo on a scale of 0-3. Table 3 shows the results of this scoring and is in agreement with the data obtained from the plate reader.

TABLE 3
Relative Transient GFP expression after
131440 co-cultivation in experiment ZM1

| | n | GFP0 | GFP1 | GFP2 | GFP3 |
|---|---|---|---|---|---|
| Untreated | 167 | 28.1% | 58.7% | 12.6% | 0.6% |
| S233 Pre- | 154 | 7.1% | 16.2% | 29.2% | 47.4% |
| conditioned | | | | | |

### Example 5—Comparison of Surfactant Pre-Conditioning Treatments on Maize Transient GFP Expression

Transient expression of GFP was measured following co-cultivation of maize tissue with LBA4404 Agrobacterium cells harboring superbinary vector 130571. The effect of pre-conditioning medium comprising 0.01% (v/v) Break-Thru S233 or Break-Thru S301 was tested. Twenty-four embryos each were selected from: 1) untreated embryos, 2) Break-Thru S233 preconditioned embryos, and

3) Break-Thru S301 preconditioned embryos. GFP fluorescence was quantified for these embryos, as summarized in Table 4.

TABLE 4
RFU Values for untreated embryos, S233 pre-conditioned,
and S301 pre-conditioned embryos in experiment ZM4

| | untreated | S233 | S301 |
|---|---|---|---|
| <500 | 11 | 8 | 7 |
| 501-999 | 4 | 0 | 4 |
| 1000-1500 | 1 | 1 | 0 |
| 1501-2000 | 2 | 5 | 0 |
| >2001 | 6 | 10 | 13 |

As Table 4 shows, pre-conditioning with either Break-Thru S233 or Break-Thru S301 resulted in an increased number of highly-expressing immature embryos (embryos with an RFU reading of >2001) relative to untreated embryos that did not receive any preconditioning treatment.

GFP expression in maize immature embryos in experiment ZM4 were also scored according to the relative GFP expression protocol, scoring each embryo on a scale of 0-3. Table 5 shows the results of this scoring which is consistent with the data obtained from the plate reader.

TABLE 5
Relative Transient GFP expression in experiment ZM4

| | n | GFP0 | GFP1 | GFP2 | GFP3 |
|---|---|---|---|---|---|
| Unconditioned | 80 | 21% | 50% | 24% | 5% |
| S233 | 100 | 9% | 15% | 39% | 37% |
| S301 | 81 | 10% | 15% | 40% | 36% |

The effect of using different surfactants for pre-conditioning was examined further. Transient expression of GFP was measured following co-cultivation of maize tissue with LBA4404 Agrobacterium cells harboring superbinary vector 130571. The effect of pre-conditioning medium comprising 0.01% (v/v) Break-Thru S233, Break-Thru S240, Break-Thru S279, or Break-Thru S301 was tested. GFP fluorescence was scored by visual inspection on a scale from 0-3, with 0 indicating no visible fluorescence and 3 indicating a high level of fluorescence. Table 6 summarizes the results of these experiments.

TABLE 6
RFU Values for control and pre-conditioned
maize embryos in experiment ZM3

| Preconditioning | n | GFP 0 | GFP 1 | GFP 2 | GFP 3 |
|---|---|---|---|---|---|
| Control | 106 | 18.9% | 51.9% | 21.7% | 7.5% |
| 233 | 87 | 1.1% | 14.9% | 32.2% | 51.7% |
| 240 | 94 | 6.4% | 28.7% | 26.6% | 38.3% |
| 279 | 104 | 5.8% | 43.3% | 19.2% | 31.7% |
| 301 | 85 | 0.0% | 9.4% | 29.4% | 61.2% |

As Table 6 shows, all four of the pre-conditioning treatments led to a decrease in the proportion of embryos that failed to show any visible fluorescence. All of the pre-conditioning treatments also led to an increase in the proportion of embryos scored as ‘3’ indicating very high levels of GFP fluorescence and to an increase in the proportion of embryos scored as ‘2’ or ‘3’ indicating high levels of GFP fluorescence (29.2%, 83.9%, 64.9%, 51.0%, and 90.6%, respectively, for control, S233, S240, S279, and S301 preconditioning treatments). This data indicates that preconditioning maize immature embryos with Break-Thru S233, Break-Thru S240, Break-Thru S279, or Break-Thru S301 results in increased levels of transient expression of GFP relative to untreated control embryos.

### Example 6—Setaria viridis Transformation Materials and Callus Induction

### Plant Materials and Callus Induction

Mature seeds of greenhouse grown Setaria viridis were stored at 4° C. for 3-6 months prior to using them for transformation. Seeds were de-coated taking special care not to damage the embryos. Seed coats and chaffe were removed by blowing away the material and separating the clean embryos from the debris. Clean seed was placed into a 50 mL tube for sterilization.

Seeds were sterilized with 70% ethanol for 1 minute, followed by one rinse with Millipore water. Sterilization followed 40 mL 20% (v/v) commercial bleach solution containing 0.17% (v/v) Tween-20, for 8 min, with inversion. Seeds were rinsed with autoclaved Millipore water five times to effectively remove all bleach from the surface.

Sterilized seeds were allowed to air dry in a laminar flow hood on top of sterilized filter paper for a minimum of 20 minutes before plating, embryo side up, onto callus induction media (SVKT) (MS salts 4.33 g/L, MS vitamins 1000× 1 mL/L, maltose 40 g/L ZnSO₄.7H₂O 35 mg/L, CuSO₄ 0.6 mg/L, 2,4-D 2 mg/L, kinetin 0.5 mg/l, Phytagel 3.5 g/L, pH 5.8). Plates were wrapped with parafilm and incubated in a low light chamber at 26° C. for 4 weeks

After 4 weeks on callus induction media SVKT, embryogenic callus was selected and transferred to fresh callus media without kinetin SVNKT media (MS salts 4.33 g/L, MS vitamins 1000× 1 mL/L., maltose 40 g/L, ZnSO₄7H₂O 35 mg/L, CuSO₄. 0.6 mg/L, 2,4D 2 mg/L, Phytagel 3.5 g/L, pH 5.8). Any non-embryogenic calli were discarded and only white compact callus was transferred. Plates were sealed with parafilm and incubated in a low light chamber at 26° C. for 10 days. Callus was then broken down into small pieces and transferred to fresh SVNKT media for bulk up 3 days prior to transformation. Plates were wrapped with parafilm and incubated in a low light chamber at 26° C. for 3 days.

Agrobacterium Culture Initiation

Glycerol stocks of Agrobacterium containing the appropriate vector were stored at ˜80° C. until ready to use. A loop from the frozen glycerol stock was streaked on AB minimal medium plates containing appropriate antibiotics and plates were grown at 20-25° C. for 3 days in the dark. A single colony was then picked and streaked onto YEP plates containing the same antibiotics and was incubated at 28° C. for 1-3 days.

Agrobacterium Culture

On the day of the experiment, Agrobacterium colonies were taken from the YEP plate, suspended in 10 mL of infection medium in a 50 mL disposable tube, and the cell density adjusted to OD600=0.2-0.4 nm for Agrobacterium LB A4404 harboring superbinary vector 130836 (SEQ ID NO 11) using a spectrophotometer. Agrobacterium cultures were placed on a rotary shaker at 120-130 rpm, room temperature, while embryo dissection was performed.

Setaria viridis Pre-Conditioning

Callus from 3-day sub-culture plates was transferred to empty 50 ml conical tubes. One plate of callus should fill one 50 ml tube. The preconditioning treatment lasted between 5 min-60 min at

Agrobacterium Infection and Co-Cultivation

After all callus was collected and preconditioned, the preconditioning media was removed and discarded. After removal of the preconditioning media, enough Agrobacterium suspension was added to each tube to cover the callus. Tubes were then vortexed on high setting for 15-20 seconds and allowed to rest at room temperature in the dark for 5 minutes. Agrobacterium suspension was then poured out onto a petri dish containing sterile filter paper (2), allowing the filter paper to soak all suspension. Explants were allowed to air dry for 5 minutes in the laminar flow hood before transferring the top filter paper to MS co-culture media MS CC (MS salts 4.33 g/L, MS vitamins (1000×) 1 mL/L, sucrose 20 g/L, glucose 10 g/L, casein 0.1 g/L, L-proline 0.7 g/L, 2,4-D 1.5 mg/L, MES 0.5 g/L, Phytagel 3.5 g/L, pH 5.8). Plates were wrapped with vent tape and incubated at 25° C. in the dark for 72 hours.

Callus Selection and Regeneration of Putative Events

Following the co-cultivation period, calli were transferred to selection 1 media SV Set 60 (MS salts 4.33 g/L, MS vitamins 1000× 1 mL/L, maltose 40 g/L., ZnSO₄7H₂O 35 mg/L, CuSO₄ 0.6 mg/L 2,4-D 2 mg/L, Timentin 10 mg/L, Hygromycin 6 mg;/L, Phytagel 3.5 g/L, pH 5.8). Plates were wrapped with parafilm and incubated in the dark at 26° C. for 14±2 days. Callus was transferred to fresh selection 2 media SV Sel 60, taking care to keep original callus pieces together. Plates were wrapped with parafilm and incubated in the dark at 26° C. for 14+2 days. After 14+2 days, explants were transferred to selection 3 media SV Sel 60+KT (MS salts 4.33 g/L, MS vitamins 1000× 1 mL/L, maltose 40 g/L, ZnSO₄7H₂O 35 mg/L, CuSO₄ 0.6 mg/L, 2,4-D 2 mg/L, kinetin 0.5 mg/L, Timentin 100 mg/L, Hygromycin 60 mg/L, Phytagel 3.5 g/L, pH 5.8). Plates were wrapped with parafilm and incubated in the dark at 26° C. for 1412 days. Callus was then transferred to Regeneration 1 media (MS salts 433 g/L, MS vitamins 1000× 1 mL/L, maltose 40 g/L, ZnSO₄.7H₂O 35 mg/L, CuSO₄ 0.6 mg/L, 2,4-D 2 mg/L, kinetin 0.2 mg/L, Tinentin 100 mg/L, Hygronycin 20 mg/L, Phytagel 3.5 g/L, pH 5.8). Once plantlet formation was established, they were transferred to a larger vessel to allow root formation to establish. Plantlets were handed off to greenhouse for acclimation and molecular characterization

### Example 7—Transient GFP Expression in Setaria viridis Following Superbinary Vector Transformation

Transient expression of GFP was measured following co-cultivation of S. viridis tissue with LBA4404 Agrobacterium cells harboring superbinary vector 130836. The effect of pre-conditioning medium comprising 0.01% (v/v) Break-Thru S233 or 0.01% (v/v) Break-Thru S301 was tested. GFP fluorescence was scored based on visual inspection. Table 7 shows the results of quantification of this transient GFP expression.

TABLE 7
Transient GFP expression after 130836 co-cultivation

| | n | GFP0 | GFP1 | GFP2 | GFP3 |
|---|---|---|---|---|---|
| Unconditioned | 76 | 26% | 59% | 13% | 1% |
| S233 | 85 | 6% | 9% | 19% | 66% |
| S301 | 87 | 13% | 17% | 33% | 37% |

As Table 7 shows, treatment with either Break-Thru S233 or Break-Thru S301 resulted in a shift toward higher expression of GFP in S. viridis tissue following infection with the 130836 vector. While 22.0-31.4% of untreated control callus pieces failed to show detectable GFP expression and only 0.0-2.4% of those untreated control callus pieces were scored as ‘3,’ indicating high GFP fluorescence, only 4.7-7.1% and 10.9-14.6% of S233 and S301-treated S. viridis callus pieces, respectively, failed to show detectable fluorescence. 64.3-67.4% and 34.1-39.1% of S233 and S301-treated S. viridis callus pieces, respectively, were scored as ‘3,’ indicating high GFP fluorescence. Untreated control S. viridis callus pieces showed just 11.4-17.1% scored as high GFP expressors (scores of ‘2’ or ‘3’), while S233 and S301 callus pieces showed 83.7-85.7% and 63.4-76.1%, respectively, that were scored as either ‘2’ or ‘3.’ These results indicate that pre-conditioning of S. viridis tissue with Break-Thru S233 or Break-Thru S301 results in increased transient GFP expression measured as the fraction of callus pieces showing visible GFP-derived fluorescence or as the fraction of callus pieces showing high levels of GFP-derived fluorescence.

### Example 8—Stable GFP Expression in Maize Following Plant Regeneration

Following co-cultivation with Agrobacterium cells harboring appropriate transformation vectors, maize immature embryos were maintained on tissue culture medium comprising appropriate selective agents to prevent growth of untransformed cells and appropriate hormones and other components to promote shoot growth. Following the appearance of shoots, these shoots were transferred to appropriate tissue culture medium for rooting. After root establishment, rooted plantlets were transferred to soil for growth in a greenhouse. Tissue samples may be collected from shoots prior to root establishment or after root establishment, when the plants are maintained on tissue culture medium or in soil. These samples are analyzed for GFP expression by visual inspection and/or by well-known molecular or biochemical methods such as Northern or western blotting or RT-PCR methods to detect RNA and/or protein accumulation of the GFP transcript and/or protein. Alternatively, GFP expression may be assessed in whole plants without collecting any samples through visual inspection. Because Agrobacterium has been eliminated from these cultures, detection of GFP expression and/or protein accumulation indicates that the GFP gene is stably inserted in the plant genome.

Tables 8, 9, and 10 summarize the quantification of stable GFP expression in immature embryos in experiments ZM1, ZM3, and ZM4, respectively.

TABLE 8
Stable GFP expression in experiment ZM1

| | # Transformed | GFP | GFP |
|---|---|---|---|
| Treatment | embryos | sectors | sectors |
| Unconditioned | 167 | 23 | 14% |
| S233 conditioned | 154 | 61 | 40% |

TABLE 9
Stable GFP expression in experiment ZM3

| | # Embryos | GFP | GFP |
|---|---|---|---|
| Treatment | transformed | sectors | sectors |
| Unconditioned | 106 | 12 | 11% |
| S233 conditioned | 87 | 50 | 57% |
| S240 conditioned | 94 | 71 | 76% |
| S279 conditioned | 104 | 30 | 29% |
| S301 conditioned | 85 | 39 | 46% |

TABLE 10
Stable GFP expression in experiment ZM4

| | | | GFP |
|---|---|---|---|
| | # Embryos | GFP | sectors |
| Treatment | transformed | sectors | (%) |
| Unconditioned | 80 | 46 | 58% |
| S233 conditioned | 100 | 78 | 78% |
| S301 conditioned | 81 | 74 | 91% |

The data in Tables 8-10 show that pre-conditioning with the tested surfactants led to an increase in the proportion of stably expressing GFP sectors relative to unconditioned maize embryos.

GFP expression was also quantified in regenerated maize plantlets produced from experiments ZM1, ZM3, and ZM4, as summarized in Tables 11-13, respectively.

TABLE 11
GFP expression in regenerated plantlets in experiment ZM1

| | | # GFP- | |
|---|---|---|---|
| | # Transformed | Positive | |
| Treatment | embryos | events | Transformation % |
| Unconditioned | 167 | 15 | 9% |
| S233 conditioned | 154 | 32 | 21% |

TABLE 12
GFP expression in regenerated plantlets in experiment ZM3

| | | # GFP | |
|---|---|---|---|
| | # Embryos | positive | |
| Treatment | transformed | events | Transformation % |
| Unconditioned | 106 | 11 | 10.4% |
| S233 conditioned | 87 | 29 | 33.3% |
| S240 conditioned | 94 | 18 | 19.1% |
| S279 conditioned | 104 | 14 | 13.5% |
| S301 conditioned | 85 | 30 | 35.3% |

TABLE 13
GFP expression in regenerated plantlets in experiment ZM4

| | | # GFP- | |
|---|---|---|---|
| | # Embryos | Positive | |
| Treatment | transformed | events | Transformation % |
| Unconditioned | 80 | 19 | 23.8% |
| S233 conditioned | 100 | 39 | 39.0% |
| S301 conditioned | 81 | 36 | 44.4% |

The data in Tables 11-13 show that pre-conditioning with the tested surfactants led to an increase in the number of GFP-positive regenerated maize plants in each of these experiments.

Following maturation of the TO generation plants, the plants are pollinated and the resulting seeds may be grown to produce T1 generation plants. These T1 generation plants are similarly analyzed for stable expression of GFP.

### Example 9—Stable GFP Expression in Setaria viridis Following Plant Regeneration

Following co-cultivation with Agrobacterium cells harboring appropriate transformation vectors, S. viridis callus pieces were maintained on tissue culture medium comprising appropriate selective agents to prevent growth of untransformed cells and appropriate hormones and other components to promote shoot growth. Following the appearance of shoots, these shoots were transferred to appropriate tissue culture medium for rooting. After root establishment, rooted plantlets were transferred to soil for growth in a greenhouse. Tissue samples may be collected from shoots prior to root establishment or after root establishment, when the plants are maintained on tissue culture medium or in soil. These samples are analyzed for GFP expression by visual inspection and/or by well-known molecular or biochemical methods such as Northern or western blotting or RT-PCR methods to detect RNA and/or protein accumulation of the GFP transcript and/or protein. Alternatively, GFP expression may be assessed visually in the plantlets without collecting any tissue samples. Because Agrobacterium has been eliminated from these cultures, detection of GFP expression and/or protein accumulation indicates that the GFP gene is stably inserted in the plant genome.

Table 14 summarizes the results of quantifying stable GFP expression in S. viridis callus tissues.

TABLE 14
Stable GFP expression in S. viridis callus

| | # Calli | GFP | GFP |
|---|---|---|---|
| Treatment | transformed | sectors | sectors |
| Unconditioned | 76 | 6 | 8% |
| S233 conditioned | 85 | 28 | 33% |
| S301 conditioned | 87 | 10 | 11% |

The data in Table 14 show that pre-conditioning S. viridis with either of the tested surfactants led to an increase in the proportion of stable sectors relative to unconditioned S. viridis.

GFP expression was also quantified in regenerated S. viridis plantlets, as summarized in Table 15.

TABLE 15
GFP expression in regenerated S. viridis plantlets

| | | # GFP- | |
|---|---|---|---|
| | # Calli | Positive | Transformation |
| Treatment | transformed | events | % |
| Unconditioned | 76 | 2 | 3% |
| S233 conditioned | 85 | 11 | 13% |
| S301 conditioned | 87 | 2 | 2% |

The data in Table 15 show that pre-conditioning with S233, but not with S301, led to an increase in the proportion of GFP-positive regenerated S. viridis plantlets relative to unconditioned S. viridis.

Following maturation of the TO generation plants, the plants are pollinated and the resulting seeds may be grown to produce T1 generation plants. These T1 generation plants are similarly analyzed for stable expression of GFP.

### Example 10—Pea Transformation Materials and Transformation Protocols

Plant Materials

Seeds Sterilization, Germination and Explant Preparation

Seeds of Yellow Pea (Pisum sativum cv. Amigo) were surface sterilized by immersion in a 30% (v/v) solution of bleach containing 0.05% (v/v) Tween-20. The seeds were shaken for 30-45 minutes, followed by three rinses in sterile water. After sterilization, seeds were cultured on MS media (MS salts, 4.33 gm/L; B5 vitamins [1000×] (Gamborg et al. 1968), 1.00 mL/L; sucrose, 30 gm/L; 2,4-D, 1-2 mg/L; pH adjusted to 5.8). Seeds were incubated in the dark for 1-2 days at 25° C. Pre-cultured seeds were then either 1) longitudinally split into two halves with each half containing part of the embryonic leaf/shoot and root targeting competent cells for transformation and regeneration (split seed explants), or 2) the embryo containing shoot and root was removed and used for transformation (meristem tissues).

Agrobacterium Culture Initiation

Glycerol stocks of Agrobacterium containing a vector 133337 (SEQ ID NO:12), comprising Cp4 (SEQ ID NO:13, encoding SEQ ID NO:14) and GFP with a C-terminal SEKDEL fusion (SEQ ID NO:15, encoding SEQ ID NO:16) genes as selectable and visual marker genes, respectively, were stored at −80° C. until ready to use. A loop from the frozen glycerol was streaked on AB minimal medium plates containing appropriate antibiotics and plates were grown at 20-25° C. for 3 days in the dark. A single colony was then picked and streaked onto YEP plate containing the same antibiotics and was incubated at 28° C. for 1-3 days.

Agrobacterium Culture, Infection, and Co-Cultivation

On the day of the experiment, a loop of Agrobacterium tumefaciens strain AGL1 harboring vector 133337 was taken from the YEP plate, suspended in 10 mL of infection medium in a 50 mL disposable tube, and the cell density at OD 600 nm was adjusted to 0.2-0.4 using a spectrophotometer. Agrobacterium culture was placed on a rotary shaker at 120-130 rpm, room temperature, while explant preparation was performed.

After a 1-2-day pre-culture period, split-seed explants were either collected in 10-15 mL of the infection medium alone or in infection media plus different surfactant agents for preconditioning. The preconditioning treatment lasted between 5 min-60 min. After all explants were transferred preconditioned media and infection media were discarded. Ten to fifteen milliliters of Agrobacterium suspension were added to each tube containing the split-seed explants. Tubes were inverted a few times and placed on rocker shaker for 30-45 minutes. After inoculation, the Agrobacterium culture was discarded, and explants were then blotted dry on sterile filter paper to remove excess inoculum. Infected split-seeds were then transferred adaxial side up onto co-culture media (MS salts, 4.33 gm/L; B5 vitamins [1000×] (Gamborg et al. 1968), 1.00 mL/L; sucrose, 30 gm/L; 2,4-D, 1 mg/L; Kinetin, 0.5 mg/L; Acetosyringone, 200 μM; Gelzan™ 2.3 gm/L; pH was adjusted to 5.6 prior to autoclaving). The plates were incubated for 3-5 days in the dark at 21-25° C.

Selection and Regeneration of Transgenic Shoots

After 3-5 days of co-cultivation, split-seed explants were blotted onto sterile filter paper and were then transferred onto selection media 1 (MS salts, 4.33 gm/L; B5 vitamins [1000×] (Gamborg et al. 1968), 1.00 mL/L; sucrose, 30 gm/L; BA, 2.0 mg/L; NAA, 0.2 mg/L; Glyphosate 0.1 mM; cefotaxime 250 mg/L; Gelzan™ 2.3 gm/L; pH was adjusted to 5.8 prior to autoclaving). Plates were cultured at 25° C., 16 h photoperiod, 50 μmol m²s⁻¹ light intensity. Explants were sub-cultured onto selection media 2 (MS salts, 4.33 gm/L; B5 vitamins [1000×] (Gamborg et al. 1968), 1.00 mL/L; sucrose, 30 gm/L; Zeatin, 2 mg/L; Kinetin, 0.5 mg/L; Glyphosate 0.1 mM; cefotaxime 250 mg/L; Gelzan™ 2.3 gm/L; pH was adjusted to 5.8 prior to autoclaving). Explants were sub-cultured every two weeks on the same media and incubated at 25° C., 16 h photoperiod, 50 μmol m²s⁻¹ light intensity until shoot regeneration.

### Example 11—Transient GFP Expression in Pea

Transient expression of GFP was measured following co-cultivation of P. sativum split seed tissue with AGL1 Agrobacterium cells harboring the 133337 vector. The effect of pre-conditioning medium comprising 0.01% (v/v) Break-Thru S233 or 0.01% (v/v) Break-Thru S301 was tested. GFP fluorescence was scored based on visual inspection. Table 16 shows the results of quantification of this transient GFP expression in split P. sativum seeds.

TABLE 16
Transient GFP expression in P. sativum
split seeds after 133337 co-cultivation

| Treatment | n | GFP 0 | GFP1 | GFP 2 | GFP 3 |
|---|---|---|---|---|---|
| Unconditioned | 40 | 0 | 35.0 | 37.5 | 27.5 |
| S301 | 42 | 0 | 4.8 | 52.4 | 42.9 |
| S233 | 40 | 0 | 12.5 | 50.0 | 37.5 |

As Table 16 shows, treatment with either Break-Thru S233 or Break-Thru S301 resulted in a shift toward higher expression of GFP in split seeds of P. sativum tissue following infection with the 133337 vector. While 35.0% of untreated control split seed pieces showed low levels of GFP expression (GFP category 1), only 12.5% and 4.8% of S233 and S301-treated P. sativum split seeds, respectively, showed these low levels of GFP fluorescence. Untreated control split seeds of P. sativum showed just 65.0% scored as high GFP expressors (scores of ‘2’ or ‘3’), while S233 and S301 callus pieces showed 87.5% and 95.2%, respectively, that were scored as either ‘2’ or ‘3.’ These results indicate that pre-conditioning of split seeds of P. sativum with Break-Thru S233 or Break-Thru S301 results in increased transient GFP expression measured as the fraction of split seeds showing visible GFP-derived fluorescence or as the fraction of split seeds showing high levels of GFP-derived fluorescence.

Transient expression of GFP was also measured following co-cultivation of P. sativum meristem tissue with AGL1 Agrobacterium cells harboring the 133337 vector. The effect of pre-conditioning medium comprising 0.01% (v/v) Break-Thru S233 or 0.01% (v/v) Break-Thru S301 was tested. GFP fluorescence was scored based on visual inspection. Table 17 shows the results of quantification of this transient GFP expression in P. sativum meristem tissues.

TABLE 17
Transient GFP expression in P. sativum
meristem tissue after 133337 co-cultivation

| Treatment | n | GFP 0 | GFP 1 | GFP 2 | GFP 3 |
|---|---|---|---|---|---|
| Unconditioned | 19 | 0 | 68.4 | 21.1 | 10.5 |
| S301 | 20 | 0 | 40.0 | 35.0 | 25.0 |
| S233 | 20 | 0 | 35.0 | 45.0 | 20.0 |

As Table 17 shows, treatment with either Break-Thru S233 or Break-Thru S301 resulted in a shift toward higher expression of GFP in meristematic tissue of P. sativum following infection with the 133337 vector. While 68.4% of untreated control split seed pieces showed low levels of GFP expression (GFP category 1), only 35.0% and 40.0% of S233 and S301-treated P. sativum meristematic tissues, respectively, showed these low levels of GFP fluorescence. Untreated control split seeds of P. sativum showed just 31.6% scored as high GFP expressors (scores of ‘2’ or ‘3’), while S233 and S301 callus pieces showed 65.0% and 60.0%, respectively, that were scored as either ‘2’ or ‘3.’ These results indicate that pre-conditioning of meristematic tissue of P. sativum with Break-Thru S233 or Break-Thru S301 results in increased transient GFP expression measured as the fraction of meristematic tissues showing visible GFP-derived fluorescence or as the fraction of meristematic tissue pieces showing high levels of GFP-derived fluorescence.

Transient expression of GFP was measured in a separate set of experiments following co-cultivation of P. sativum split seed tissue with AGL1 Agrobacterium cells harboring the 133337 vector. The effect of pre-conditioning medium comprising 0.01% (v/v) Break-Thru S301 or 0.01% (v/v) Pluronic™ F-68 was tested. GFP fluorescence was scored based on visual inspection. Table 18 shows the results of quantification of this transient GFP expression in split P. sativum seeds.

TABLE 18
Transient GFP expression in P. sativum
split seeds after 133337 co-cultivation

| Treatment | n | GFP 0 | GFP 1 | GFP 2 | GFP 3 |
|---|---|---|---|---|---|
| Unconditioned | 40 | 0 | 35.0 | 30.0 | 35.0 |
| S301 | 40 | 0 | 5.0 | 45.0 | 50.0 |
| Pluronic | 40 | 0 | 5.0 | 42.5 | 52.5 |

As Table 18 shows, preconditioning with either Break-Thru S301 or with Pluronic resulted in a shift toward higher expression of GFP in split seeds of P. sativum tissue following infection with the 133337 vector. Further, the data in Table 8 shows that the effect of preconditioning with Break-Thru S301 is reproducible based on a comparison with the data in Table 6. While 35.0% of untreated control split seed pieces showed low levels of GFP expression (GFP category 1), only 5.0% of S301 or Pluronic-preconditioned P. sativum split seeds, respectively, showed these low levels of GFP fluorescence. Unconditioned control split seeds of P. sativum showed just 65.0% scored as high GFP expressors (scores of ‘2’ or ‘3’), while S301 and Pluronic-preconditioned callus pieces showed 95.0% that were scored as either ‘2’ or ‘3.’ These results indicate that pre-conditioning of split seeds of P. sativum with Break-Thru S301 or Pluronic results in increased transient GFP expression measured as the fraction of split seeds showing visible GFP-derived fluorescence or as the fraction of split seeds showing high levels of GFP-derived fluorescence.

Transient expression of GFP was also measured in a separate set of experiments following co-cultivation of P. sativum meristem tissue with AGL1 Agrobacterium cells harboring the 133337 vector. The effect of pre-conditioning medium comprising 0.01% (v/v) Break-Thru S301 or 0.01% (v/v) Pluronic™ F-68 was tested. GFP fluorescence was scored based on visual inspection. Table 19 shows the results of quantification of this transient GFP expression in P. sativum meristem tissues.

TABLE 19
Transient GFP expression in P. sativum
meristem tissue after 133337 co-cultivation

| Treatment | n | GFP 0 | GFP 1 | GFP 2 | GFP 3 |
|---|---|---|---|---|---|
| Unconditioned | 20 | 0 | 60.0 | 30.0 | 10.0 |
| S301 | 20 | 0 | 30.0 | 45.0 | 25.0 |
| Pluronic | 14 | 0 | 35.7 | 35.7 | 28.6 |

As Table 19 shows, preconditioning with either Break-Thru S301 or with Pluronic resulted in a shift toward higher expression of GFP in meristematic tissue of P. sativum following infection with the 133337 vector. Comparison of the data in Table 9 with the data in Table 7 also shows that preconditioning with Break-Thru S301 is reproducible. In these experiments, while 60.0% of untreated control split seed pieces showed low levels of GFP expression (GFP category 1), only 30.0% and 35.7% of S301 and Pluronic-preconditioned P. sativum meristematic tissues, respectively, showed these low levels of GFP fluorescence. Untreated control split seeds of P. sativum showed just 40.0% scored as high GFP expressors (scores of ‘2’ or ‘3’), while S301 and Pluronic callus pieces showed 70.0% and 64.3%, respectively, that were scored as either ‘2’ or ‘3.’ These results indicate that pre-conditioning of meristematic tissue of P. sativum with Break-Thru S301 or Pluronic results in increased transient GFP expression measured as the fraction of meristematic tissues showing visible GFP-derived fluorescence or as the fraction of meristematic tissue pieces showing high levels of GFP-derived fluorescence.

### Example 12—Tomato Transformation Materials and Transformation Protocols

Plant Materials

Seeds Sterilization, Germination and Explant Preparation

Seeds of tomato (Solanum lycopersicum cv. Rio Grande) were surface sterilized by immersion in a 20% (v/v) solution of household bleach containing 0.25% (v/v) Tween-20. The seeds were shaken for 20 minutes, followed by three rinses in sterile water. After sterilization, seeds were germinated in phytatrays (Sigma-Aldrich, St. Louis, Mo.) containing ½X MS media (½X MS salts, 2.17 gm/L; B5 vitamins [1000×] (Gamborg et al. 1968), 1.00 mL/L; sucrose, 15 gm/L, pH was adjusted to 5.8). Seeds were incubated in the dark for 2-3 days at 25° C. Germinated seeds were then transferred to a lit chamber (16 h photoperiod, 45 μmol m²s⁻¹ light intensity and 60% relative humidity) for 8-13 days. For a given set of experiments, pooled cotyledons from 8-13 day-old seedlings were cut and precultured on MS media (MS salts, 4.33 gm/L; B5 vitamins [1000×](Gamborg et al. 1968), 1.00 mL/L; sucrose, 30 gm/L; BA, 1.5 mg/L; NAA, 0.1 mg/L; Gelzan™ 2.3 gm/L, pH adjusted to 5.8 prior to autoclaving). Plates were cultured in the dark or a lit chamber (16 h of photoperiod, 45 μmol m²s⁻¹ light intensity and 60% relative humidity (RH)) at 25° C.

Agrobacterium Culture Initiation

Glycerol stocks of Agrobacterium containing vector 133336 (SEQ ID NO:17), which comprises NptII (SEQ ID NO:3, encoding SEQ ID NO:4) and GFP with a C-terminal SEKDEL fusion (SEQ ID NO:15, encoding SEQ ID NO:16) genes as selectable marker and visual selection genes, respectively were stored at −80° C. until ready to use. A loop from the frozen glycerol stock was streaked on AB minimal medium plates containing appropriate antibiotics and plates were grown at 20-25° C. for 3 days in the dark. A single colony was then picked and streaked onto a YEP plate containing the same antibiotics and was incubated at 28° C. for 1-3 days.

Agrobacterium Culture, Infection, and Co-Cultivation

On the day of the experiment, a loop of Agrobacterium AGL1/p133336 was taken from the YEP plate, suspended in 10 mL of infection medium in a 50 mL disposable tube, and the cell density at OD 600 nm was adjusted to 0.2-0.4 for AGL1 using a spectrophotometer. Agrobacterium culture was placed on a rotary shaker at 120-130 rpm at room temperature, while explant preparation was performed.

After 2-day pre-culture period, cotyledon explants were either collected in 5-10 mL of the infection medium alone or in infection media comprising the appropriate surfactant agent for preconditioning. The preconditioning treatment lasted between 5 min-60 min. After preconditioning, the preconditioning media and infection media were discarded and ten milliliters of Agrobacterium suspension were added to each tube. Tubes were inverted a few times and placed on rocker shaker for 15-30 minutes. After inoculation, Agrobacterium culture was discarded, and explants were then blotted dry on sterile filter paper to remove excess inoculum. Infected cotyledons were then transferred abaxial side up onto co-culture media (MS salts, 4.33 gm/L; B5 vitamins [1000×] (Gamborg et al. 1968), 1.00 mL/L; sucrose, 30 gm/L; BA, 1.5 mg/L; NAA, 0.1 mg/L; Acetosyringone, 200 μM; Gelzan™ 2.3 gm/L, pH adjusted to 5.8 prior to autoclaving). The plates were incubated for 2-3 days in the dark at 21-25° C.

Selection and Regeneration of Transgenic Shoots

After 2-3 days of co-cultivation, the explants were washed thoroughly with MS liquid media containing cefotaxime (150 mg/L) to remove excess inoculum. Explants were blotted onto sterile filter paper and were then transferred onto selection media (MS salts, 4.33 gm/L; B5 vitamins [1000×] (Gamborg et al. 1968), 1.00 mL/L; sucrose, 30 gm/L; Zeatin, 2.0 mg/L; IAA, 0.5 mg/L; Kanamycin 50-100 mg/L and cefotaxime 150 mg/L; Gelzan™ 2.3 gm/L, pH adjusted to 5.8 prior to autoclaving). Plates were cultured at 25° C., 16 h photoperiod, 50 μmol m²s⁻¹ light intensity and 50% RH. Explants were sub-cultured onto fresh selection media every two weeks until shoot regeneration.

### Example 13—Transient GFP Expression in Tomato

Transient expression of GFP was measured following co-cultivation of S. lycopersicum cotyledon tissue with AGL1 Agrobacterium cells harboring the 133336 vector. The effect of pre-conditioning medium comprising 0.01% (v/v) Break-Thru S233 or 0.01% (v/v) Break-Thru S301 was tested with the cotyledons of eight day-old seedlings. GFP fluorescence was scored based on visual inspection. Table 20 shows the results of quantification of this transient GFP expression in S. lycopersicum cotyledon tissue.

TABLE 20
Transient GFP expression in cotyledon tissue from 8-day old
S. lycopersicum seedlings after 133336 co-cultivation

| Treatment | n | GFP 0 | GFP 1 | GFP 2 | GFP 3 |
|---|---|---|---|---|---|
| Unconditioned | 50 | 12.0 | 56.0 | 16.0 | 16.0 |
| S233 | 52 | 7.7 | 19.2 | 46.2 | 26.9 |
| S301 | 55 | 5.5 | 12.7 | 52.7 | 29.1 |

As Table 20 shows, preconditioning treatment with either Break-Thru S233 or Break-Thru S301 resulted in a shift toward higher expression of GFP in cotyledon tissue of S. lycopersicum following infection with the 133336 vector. While 12.0% and 56.0% of untreated control cotyledon tissue pieces showed no GFP expression or low levels of GFP expression (GFP category 0 or 1, respectively), respectively, only 7.7% and 5.5% of S233 and S301-treated S. lycopersicum cotyledon tissues, respectively, showed undetectable levels of GFP fluorescence, and only 19.2% and 12.7% of S233 and S301-preconditioned S. lycopersicum cotyledon tissues, respectively, showed low levels of GFP expression (category 1). Untreated control cotyledon tissue showed just 32.0% scored as high GFP expressors (scores of ‘2’ or ‘3’), while S233 and S301 callus pieces showed 73.1% and 81.8%, respectively, that were scored as either ‘2’ or ‘3.’ These results indicate that pre-conditioning of cotyledon tissue of S. lycopersicum with Break-Thru S233 or Break-Thru S301 results in increased transient GFP expression measured as the fraction of cotyledon tissues showing visible GFP-derived fluorescence or as the fraction of cotyledon tissue pieces showing high levels of GFP-derived fluorescence.

Transient expression of GFP was measured following co-cultivation of S. lycopersicum cotyledon tissue with AGL1 Agrobacterium cells harboring the 133336 vector. The effect of pre-conditioning medium comprising 0.01% (v/v) Break-Thru S301 or 0.01% (v/v) Pluronic F-68 was tested with the cotyledons of ten day-old seedlings. GFP fluorescence was scored based on visual inspection. Table 21 shows the results of quantification of this transient GFP expression in S. lycopersicum cotyledon tissue.

TABLE 21
Transient GFP expression in cotyledon tissue from 10-day old
S. lycopersicum seedlings after 133336 co-cultivation

| Preconditioning | n | GFP 0 | GFP 1 | GFP 2 | GFP 3 |
|---|---|---|---|---|---|
| Unconditioned | 39 | 10.3 | 51.3 | 30.8 | 7.7 |
| S301 | 41 | 2.4 | 31.7 | 46.3 | 19.5 |
| Pluronic | 45 | 4.4 | 42.2 | 40.0 | 13.3 |

As Table 21 shows, preconditioning treatment with either Break-Thru S301 or Pluronic F-68 resulted in a shift toward higher expression of GFP in cotyledon tissue of S. lycopersicum following infection with the 133336 vector. While 10.3% and 51.3% of untreated control cotyledon tissue pieces showed no GFP expression or low levels of GFP expression (GFP category 0 or 1, respectively), respectively, only 2.4% and 31.7% of S301 and Pluronic-preconditioned S. lycopersicum cotyledon tissues, respectively, showed undetectable levels of GFP fluorescence, and only 31.7% and 42.2% of 5301—and Pluronic-preconditioned S. lycopersicum cotyledon tissues, respectively, showed low levels of GFP expression (category 1). Untreated cotyledon tissue showed just 38.5% scored as high GFP expressors (scores of ‘2’ or ‘3’), while 5301 and Pluronic-preconditioned callus pieces showed 65.9% and 53.3%, respectively, that were scored as either ‘2’ or ‘3.’ These results indicate that pre-conditioning of cotyledon tissue of S. lycopersicum with Break-Thru S301 or Pluronic F-68 results in increased transient GFP expression measured as the fraction of cotyledon tissues showing visible GFP-derived fluorescence or as the fraction of cotyledon tissue pieces showing high levels of GFP-derived fluorescence.

### Example 14—Stable GFP Expression in Pea

As transient GFP expression in pea was shown to be improved by the addition of surfactant pre-conditioning, stable GFP expression in pea transformants was investigated. Table 22 shows the transient GFP expression results from this experiment:

TABLE 22
Transient GFP expression in pea

| Treatment | GFP 0 | GFP 1 | GFP 2 | GFP 3 |
|---|---|---|---|---|
| Unconditioned | 0 | 4 | 8 | 3 |
| S233 | 0 | 3 | 7 | 5 |
| S301 | 0 | 2 | 7 | 6 |
| Pluronic F-68 | 0 | 2 | 6 | 7 |

The results shown in Table 22 show that preconditioning with S233, 5301, or Pluronic F-68 results in an increased fraction of split seeds showing high levels of GFP fluorescence. These split seeds were further cultured to produce shoots. Table 23 shows the results of this culturing and production of stably transformed plants.

TABLE 23
Stable GFP expression in pea

| Treatment | GOI+ Events | TF % |
|---|---|---|
| Unconditioned | 1 | 6.7 |
| S233 | 2 | 13.3 |
| S301 | 3 | 20 |
| Pluronic F-68 | 5 | 33.3 |

The data in Table 23 shows that preconditioning with S233, S301, or Pluronic F-68 results in an increased transformation frequency relative to unconditioned pea split seeds, with Pluronic F-68 preconditioning leading to the highest observed transformation frequency.

### Example 15—Stable GFP Expression in Tomato

As transient GFP expression in tomato was shown to be improved by the addition of surfactant pre-conditioning, stable GFP expression in pea transformants was investigated. Table 24 shows the transient GFP expression results from this experiment.

TABLE 24
Transient GFP expression in tomato

| Treatment | GFP 0 | GFP 1 | GFP 2 | GFP 3 | Total |
|---|---|---|---|---|---|
| Unconditioned | 0 | 61 | 42 | 48 | 151 |
| S301 Pre-conditioned | 0 | 17 | 55 | 70 | 142 |

The data in Table 24 shows that preconditioning with S301 results in a shift toward higher levels of transient GFP expression. This tissue was cultured to generate stably transformed plants. Table 25 summarizes the results of this culturing and plant regeneration.

TABLE 25
Stable GFP expression in tomato

| Treatment | # Confirmed shoots | TF % |
|---|---|---|
| Unconditioned | 34 | 22.5% |
| S301 Pre-conditioned | 54 | 38.0% |

The data in Table 25 shows that S301 preconditioning leads to a substantial increase in tomato transformation efficiency.

### Example 16—Preconditioning to Enhance Biolistic Plant Transformation

Maize (Zea mays cv. B104) plant tissue was prepared for biolistic transformation essentially as described previously, with modifications (Raji et al (2018) Methods Mol Biol 1676:15-40). Immature maize embryos were transformed without a preconditioning step or with a preconditioning step comprising S301 surfactant (0.01% (v/v), 30 min following osmotic treatment). Following biolistic bombardment of the maize immature embryos with the desired DNA constructs (the “introduced DNA”), the bombarded embryos were maintained on appropriate tissue culture medium to allow for shoot regeneration and event recovery. The number of unique events comprising the introduced genes (i.e., the selectable marker gene and/or additional gene(s) of interest as appropriate) produced from these embryos following bombardment were counted, as were the number of plants. The number of plants produced included both unique events as well as sibling events produced from the same immature embryo. Sibling events may be valuable for example in genome editing experiments where the introduced DNA comprises one or more genome editing nucleases, base editors, or other genes encoding proteins capable of modifying DNA at another site or sites in the targeted genome. In such experiments, sibling plants may comprise the same introduced DNA, but may comprise different genomic modifications as a result of the action of the one or more genome editing nucleases and/or genome editing enzymes. Additionally, for genome editing, events in which one or more components for genome editing are missing but the intended editing is observed can also be useful and desired for downstream applications and would be valuable. Table 26 summarizes the results of these maize biolistic experiments.

TABLE 26
Biolistic maize transformation

| | #IE | Events | Plants | Event Freq | Plant Freq |
|---|---|---|---|---|---|
| Unconditioned | 1313 | 286 | 958 | 21.8% | 73.0% |
| S301 | 1340 | 424 | 1583 | 31.6% | 118.1% |

The data in table 26 shows that preconditioning with S301 results in substantial increases in both the number of unique events as well as the number of plants. In the absence of preconditioning, 286 events and 958 plants were produced from 1313 immature embryos (3.35 plants per event). Following S301 preconditioning, 424 unique events and 1583 plants were produced from 1340 immature embryos (3.73 plants per event). These results show that preconditioning improves plant transformation and plant regeneration efficiency using the biolistic bombardment method.

### Example 17—Preconditioning to Improve Genome Editing Results

Rice (Oryza sativa) immature embryos were transformed biolistically with and without a preconditioning treatment. The transformation vectors used in these experiments comprised a Cpf1 genome editing nuclease (vector 133869; SEQ ID NO:18) and a guide RNA (gRNA) (vector 133432; SEQ ID NO:19) along with a third vector comprising a hygromycin resistance gene (vector 131592; SEQ ID NO:20). Immature embryos were either unconditioned prior to bombardment with these vectors or were pre-conditioned with S301 surfactant (0.01% (v/v), 30 min). To test the effect of preconditioning on genome editing, DNA was extracted from the immature embryos following bombardment and the DNA was analyzed by next-generation sequencing (NGS). The immature embryos were either bombarded once (single shot) or twice (double shot). Table 27 shows the results of this NGS analysis.

TABLE 27
Rice genome editing with and without preconditioning

| | Unconditioned | Preconditioned |
|---|---|---|
| Single Shot | 0.686% | 15.216% |
| Double Shot | 0.839% | 24.177% |
| Double Shot | 11.992% | 25.489% |

The data in Table 27 show that preconditioning of rice immature embryos leads to improved genome editing results, with an increased proportion of cells comprising DNA modifications at the desired loci as indicated by the increased number of sequence reads showing DNA sequence modifications at the predicted Cpf1 cut site.

### Example 18. Addition of Surfactant to Co-Culture Media Improves Agrobacterium-Mediated

### Transformation of Immature Maize Embryos

Maize embryo cells and Agrobacterium were grown and prepared for transformation in multiple parallel workflows. In certain workflows, the plant cells were exposed to surfactant only during a preconditioning step before inoculation with Agro. In other workflows, surfactants were withheld until the plant cells had been infected with Agrobacterium and the two cell types were being grown as a coculture. In other workflows, plant cells were exposed to surfactant a first time during a preconditioning step, then removed from the surfactant, infected with Agrobacterium, and then placed onto coculture media containing surfactant. Thus the effects of exposing only the plant cells to surfactant only after infection, exposing only the plant cells to surfactant before infection, and exposing only the plant cells to surfactant a first time before infection and then a second time after infection and during coculture could be compared.

When added during coculture, the surfactant was added to the coculture media after autoclaving and the combination was mixed vigorously to ensure the viscous surfactant was fully homogenized in the coculture media.

The coculture step comprised placing the plant embryos onto the surfactant-containing media after infection and incubating. Coculture incubation, GFP expression assessments, and selection steps were performed as described herein.

Two or three different concentrations of the surfactant Break-Thru S301 in the preconditioning and coculture medias were tested. Tables 28-30 reveal the observed effects the different S301 concentrations had on transformation efficiencies.

TABLE 28
Transient GFP expression results.

| | | N | | | | |
|---|---|---|---|---|---|---|
| S301 | | (# calli | | | | |
| in pre- | S301 in | in | % GFP | % GFP | % GFP | % GFP |
| conditioning | Co-culture | selection) | 0 | 1 | 2 | 3 |
| 0% | 0.0002% | 483 | 3% | 39% | 45% | 14% |
| 0% | 0.001% | 393 | 2% | 31% | 48% | 20% |
| 0% | 0.002% | 94 | 11% | 30% | 37% | 22% |
| 0.01% | 0.0002% | 458 | 3% | 34% | 38% | 25% |
| 0.01% | 0.001% | 266 | 0% | 23% | 47% | 31% |
| 0.01% | 0.002% | 90 | 0% | 9% | 27% | 64% |

TABLE 29
Stable GFP expression results.

| | S301 | N (# | | Average |
|---|---|---|---|---|
| S301 in pre- | in Co- | calli in | GFP | GFP |
| conditioning | culture | selection) | sectors | sectors (%) |
| 0% | 0% | 90 | 14 | 16% |
| 0% | 0.0002% | 413 | 173 | 34% |
| 0% | 0.001% | 344 | 174 | 51% |
| 0% | 0.002% | 33 | 11 | 33% |
| 0.01% | 0% | 311 | 170 | 58% |
| 0.01% | 0.0002% | 375 | 218 | 61% |
| 0.01% | 0.001% | 122 | 63 | 42% |
| 0.01% | 0.002% | 1 | 0 | 0% |

TABLE 30
Stable GFP expression in regenerated plantlets.

| S301 | S301 | Transformation |
|---|---|---|
| in pre- | in Co- | Frequency |
| Treatment | culture | (TF %) |
| 0% | 0% | 1% |
| 0% | 0.0002% | 4% |
| 0% | 0.001% | 5% |
| 0% | 0.002% | 1% |
| 0.01% | 0% | 4% |
| 0.01% | 0.0002% | 5% |
| 0.01% | 0.001% | 5% |
| 0.01% | 0.002% | 0% |

These results show that exposing plant cells to surfactant during preconditioning treatment, removing the plant cells from surfactant, and then exposing them to surfactant again after infection during co-culture improves transformation efficiencies. These results also show that exposing the plant cells to surfactant only after infection during coculture also improves transformation efficiencies. It is anticipated that withholding surfactants until even later steps in the transformation process will also improve transformation efficiencies, for example, adding surfactants during the various steps of transgenic selection, callus induction, shoot elongation, regeneration, and/or rooting, etc.

### Example 19. Adding Surfactant During the Seed Imbibition Step Improves Agrobacterium-Mediated Transformation of Soybean

The effects of exposing soybean explants to surfactant only as a preconditioning treatment, as described in Example 1, were compared to the effects of exposing soybean plants to surfactant only during the seed imbibition step. A first group of soybean plants were exposed to the surfactant 0.1% Break-Thru S233 only as a preconditioning treatment after explants were prepared, as described in Example 1. Following removal from surfactant and then Agrobacteria infection, this first group of soybean plants were processed through coculture and recovery and then transformation efficiencies were assessed comprising methods described herein.

A second group of soybean plants were exposed to surfactant only during the seed imbibition step in a 0.01% solution of Break-Thru S233. Following this surfactant-imbibition step, the second group of soybean plants were removed from the surfactant, explants were prepared, and the explants were processed through infection by Agrobacteria, coculture, and recovery steps and then transformation efficiencies were assessed comprising methods described herein.

Table 31 reveals the surprising result that transformation efficiencies were improved in the plant cells treated with surfactant only during the seed imbibition step.

TABLE 31
Effect of addition of S233 during seed
imbibition on soybean transformation

| | Transformation |
|---|---|
| S233 in seed imbibition | Frequency (TF %) |
| 0% | 7.0% |
| 0.01% | 11.3% |

### Example 20. Adding Surfactant During the Seed Imbibition and Coculture Improves Agrobacterium-Mediated Transformation of Soybean

The effects of exposing soybean plants to surfactant at two steps of the transformation process, imbibition and cocultivation, were tested. A first group of soybean explants were transformed with Agrobacterium but were only exposed to surfactant as a preconditioning treatment after explants were prepared, as described in Example 1. A second group of soybean plants were also transformed with Agrobacterium, but were exposed to surfactant at two steps; the first during the seed imbibition step and the second during co-cultivation, each time with 0.01% Break-Thru S233. In both treatments, the plant cells were removed from surfactant before being exposed to the Agrobacterium.

The consequences of the two different treatments on transformation efficiency was then assessed and the results are shown in Table 32.

TABLE 32
Effect of addition of S233 during seed imbibition
and co-cultivation on soybean transformation

| | |
|---|---|
| Addition of S233 | Transformation |

| Seed imbibition | Co-cultivation | Frequency (TF %) |
|---|---|---|
| 0% | 0% | 22.0% |
| 0.01% | 0.01% | 30.0% |

This reveals that exposing plant cells to surfactant during imbibition, and then again during co-cultivation, improves transformation efficiencies dramatically.

### Example 21. Exposing Plant Cells to Surfactant Will Improve the Transformation Efficiencies of Viral-Mediated Transformation Methods

It is anticipated that plant cells can be exposed to surfactant prior to, during, and/or after the viral infection step of a virus-based transformation procedure to improve transformation efficiencies in ways that are analogous to the bacterial infection based methods described herein. Using these methods, the optimal timing and concentration parameters of adding surfactant in a viral infection based transformation process can be determined.

For example, as described herein with bacteria, a scientist can test whether exposing plant cells, including protoplasts, to a concentration of between about 0.0001% and 0.1% surfactant before, during, or after the plant cells are infected with virus and assay the effects on transformation efficiencies to determine which treatments provide the best results. Exposing the virus to surfactant prior to infection will not be necessary to improve transformation efficiencies based on the same principles disclosed herein. It is anticipated that these teachings will dramatically improve viral-mediated transformation of monocots.

### Example 22. Exposing Plant Cells to Surfactant Will Improve Transformation Efficiencies of Electroporation and Similar Methods

Results described herein support the idea that surfactant-based improvements in plant transformation efficiencies is due largely to the effects the surfactants have on the plant cells, independent of the effects they might have on an infecting bacterium. Thus, it is anticipated that plant cells can be conditioned for transformation by exposing them to surfactant before, during, or after substantially any transformation step wherein plant cells are conditioned to improve their uptake of macromolecules like polynucleotides across their cell membranes and/or cell walls.

For example, it is anticipated that plant cells can be conditioned for transformation by electroporation by exposing them to surfactant before, during, or after the electroporation step. Using methods analogous to those described herein for bacterial infection based transformation, a scientist can test whether exposing plant cells, including protoplasts, to a concentration of between about 0.0001% and 0.1% surfactant before, during, or after the plant cells are electroporated and assay the effects on transformation efficiencies to determine which surfactant treatments provide the best results.

Similarly, it is anticipated that the teachings described herein can be applied to improve the efficiencies of other transformation methods, for example but not limited to, infiltration, silicon carbide-mediated, electroporation, microinjection, polyethylene glycol (PEG), and liposome (lipofection)-based transformation methods.

### Example 23. Delivery of Proteins and Other Macromolecules into Plant Cells is Mediated by Treating Plant Cells with Surfactants

One of ordinary skill in the art will recognize that the effects surfactants have on plant cell membranes and/or cell walls that are taught herein will also make it easier to deliver macromolecules other than polynucleotides into plant cells, including proteins and other substances known to interact with biological systems that affect plant cell division, reproduction, genetics, gene expression, growth, transformation, recovery, etc.

Methods previously described demonstrate that it is possible to “transform” plant cells by delivering proteins directly into plant protoplasts (Woo, J. W. et al., Nat. Biotechnol. 33, 1162-1164 (2015); Bart, R.et al. Plant Methods 2, 13 (2006); Silva, A., et al., BMC Plant Biol. 10, 291 (2010)) and/or cells instead of delivering nucleic acids that encode such proteins and relying on the plant cells to express the genes encoded by the delivered nucleic acids. Methods of conditioning plant cells with surfactants taught herein can be used to deliver such proteins into plant cells, for example, delivering proteins capable of creating desired edits in the polynucleotides of the plant cells, e.g. genome editing proteins associated with CRISPR-like activities. For example, it has been demonstrated that Cre recombinase proteins can be delivered into plant cells to achieve nucleic acid-free genome edits in plants possessing a cell wall. (Furuhata, Y., et al., Sci Rep 9, 2163 (2019)) One of skill in the art will appreciate that the teachings herein can be applied to facilitate this type of transfer and can be applied to practically any other type of protein a user desires to deliver into a plant cell.

Thus, it follows that these methods of conditioning plant cells can be used in conjunction with any method of exposing polynucleotides, proteins, or other molecules/objects to the surface of plant cells to improve the uptake of a desired chemical by the plant cells. Examples include treating plant meristem cells of a germinated plant with surfactants before, during, or after the plant cells are exposed to a molecule the user desires to deliver into a plant cell, for example, by using methods described in Atkins and Voytas (Curr Opin Plant Biology Vol. 54, April 2020, Pages 79-84), to produce new tissues containing a desired genetic sequence.

Nor are the teachings described herein limited to delivering certain types of molecules into the cells of plants and can be used in conjunction with molecules that cause genetic mutations (e.g. genome editing components like CRISPRs) as well as molecules like DNA binding proteins, methylated oligos, RNAs, chimeric proteins and other molecules capable of altering gene expression (e.g. through methylation, acetylation, hybridization, etc.

### Example 24. Whole Seed Transformation with Surfactants

Four groups of 360 seeds each were surface sterilized using standard methods of the art and stored in sterile conditions overnight. Following sterilization all seeds were imbibed in imbibition media overnight and then incubated in an INF media supplemented with Acetosyringone with or without conditioning agent for 20 min. All the groups were removed from their imbibition/surfactant solutions. Next, one of the conditioned groups and one of the non-conditioned groups were subjected to sonication for 2 min and subjected to the otherwise standard Agrobacterium-mediated transformation methods described herein or in the art. Agrobacterium-mediated transformation was carried out using a construct containing the GFP as the visual maker and nptII or spectinomycin resistance gene as the selection marker.

After approximately 66-94 days the number of plantlets that survived were counted and recorded as putative transformation events. Leaf punch samples from each putative event (i.e. each surviving plant) were then collected and genotyped to confirm molecularly that the DNA sequence was successfully incorporated into the plant. Those plants that were confirmed to contain the transformed DNA were recorded as “Confirmed Events”. Table 33 summarizes the experimental treatments and results of these whole seed transformation experiments.

TABLE 33

| | | | Whole | Putative | Confirmed |
|---|---|---|---|---|---|
| Treatment | Conditioning | Sonication | Seeds | Events | Events |
| 1 | Yes | Yes | 360 | 40 | 2 |
| 2 | Yes | No | 360 | 8 | 0 |
| 3 | No | Yes | 360 | 8 | 1 |
| 4 | No | No | 360 | 8 | 0 |

We claim:

1. A method of transforming a plant cell with a polynucleotide by exposing the plant cell to a surfactant before the plant cell is infected by a bacterium containing the polynucleotide, or a virus containing the polynucleotide, wherein the bacterium or virus is capable of transforming the plant cell by infecting the plant cell.

2. The method of claim 1 wherein exposing the plant cell to a surfactant comprises exposing a seed comprising the plant cell to surfactant.

3. The method of claim 2 further comprising removing the plant cell from surfactant before exposing the plant cell to the bacterium or virus.

4. The method of claim 2 further comprising exposing the plant cell to an additional treatment of surfactant after the plant cell has been infected by the bacterium or virus.

5. The method of claim 4 wherein the additional treatment of surfactant is applied at least 1 hour after the plant cell has been exposed to the bacterium or virus.

6. The method of claim 2 wherein the surfactant is applied during a co-culture step.

7. The method of claim 2 wherein the surfactant is applied during a step selected from the group comprising transgenic selection, callus induction, shoot elongation, regeneration, and rooting.

8. The method of claim 2 wherein the seed is exposed to the solution for at least 4 hours.

9. A method of transforming a non-conditioned plant cell with a polynucleotide by exposing the non-conditioned plant cell to a surfactant after the non-conditioned plant cell is infected by a bacterium containing the polynucleotide, or a virus containing the polynucleotide, wherein the bacterium or virus is capable of transforming the plant cell by infecting the plant cell.

10. The method of claim 9 wherein exposure of the plant cell to surfactant occurs during a co-culture step.

11. The method of claim 9 wherein exposure of the plant cell to surfactant occurs after a co-culture step.

12. The method of any one of claims 1-11 wherein the bacterium is a species selected from the group comprising Agrobacterium, Ochrobactrum, Rhizobium, Ensifer and the collective bacterial strains termed Transbacter™.

13. A method of transforming a plant cell with a nucleotide sequence by a biolistic transformation procedure that comprises exposing the plant cell to a surfactant and wherein the biolistic transformation procedure comprises a bombardment step wherein the plant cell is bombarded by a particle capable of transforming the plant cell with the nucleotide sequence.

14. The method of claim 13 wherein the plant cell is exposed to a surfactant before the bombardment step.

15. The method of claim 13, wherein the plant cell is exposed to a surfactant during or after the bombardment step.

16. The method of claim 13 wherein the plant cell is exposed to a surfactant during at least two steps of the biolistic transformation process.

17. A method of transforming a plant cell with a macromolecule by a biolistic transformation procedure that comprises exposing the plant cell to a surfactant and wherein the biolistic transformation procedure comprises a bombardment step wherein the plant cell is bombarded by a particle capable of transforming the plant cell with the nucleotide sequence.

18. The method of claim 17 wherein the plant cell is exposed to a surfactant before the bombardment step.

19. The method of claim 17 wherein the plant cell is exposed to a surfactant during or after the bombardment step.

20. A method of transforming a plant cell by an electroporation treatment capable of delivering a polynucleotide into a plant and wherein the plant cell is exposed to a surfactant.

21. The method of claim 20 wherein the plant cell is subjected to the electroporation treatment during or after it is exposed to a surfactant.

22. The method of claim 21 wherein the plant cell is exposed to a surfactant before the electroporation treatment.

23. A method of transforming a plant cell with a macromolecule by an electroporation treatment capable of delivering the macromolecule into a plant and wherein the plant cell is exposed to a surfactant.

24. The method of claim 23 wherein the plant cell is exposed to a surfactant before the electroporation treatment.

25. The method of claim 23 wherein the plant cell is exposed to the surfactant during or after the electroporation treatment.

26. The methods of any of the claims 1-25 wherein the surfactant is in a solution at a concentration from about 0.0001% to about 0.1%.

27. The method of claims 17 or 23 wherein the macromolecule is a polypeptide.

28. The method of claim 27 wherein the macromolecule is a peptide comprising at least one CRISPR nuclease.

29. The method of claims 17 or 23 wherein the macromolecule is a ribonucleic protein.

30. The method of claim 27 wherein the polypeptide is capable of creating mutations in a nucleotide sequence of the plant cell.

31. The method of claim 27 or 30 wherein the polypeptide comprises at least one CRISPR nuclease.

32. A method of improving transformation of plant cells comprising:
i) pre-conditioning plant cells by exposure to a surfactant containing medium,
ii) removing said plant cells from said surfactant containing medium, and
iii) introducing at least one polynucleotide sequence into said plant cells.

33. The method of claim 32, wherein said surfactant containing medium comprises a non-ionic surfactant.

34. The method of claim 32, wherein said surfactant containing medium comprises a surfactant selected from the group consisting of Break-Thru S233, Break-Thru S240, Break-Thru S279, Break-Thru S301, and Pluronic F-68.

35. The method of claim 32, wherein said surfactant containing medium comprises surfactant at a concentration of 0.001-0.1% (v/v).

36. The method of claim 32 wherein said exposure to a surfactant containing medium lasts for 5-60 minutes.

37. The method of claim 32 wherein said introducing one or more polynucleotide sequence(s) includes the use of Agrobacterium cells harboring a plant transformation construct.

38. The method of claim 37 wherein said Agrobacterium cells harboring a plant transformation construct comprise a binary vector.

39. The method of claim 37 wherein said Agrobacterium cells harboring a plant transformation construct comprise a superbinary vector.

40. The method of claim 32 wherein said improving transformation of plant cells comprises an increased percentage of plant cells exhibiting transient expression of said at least one polynucleotide sequence relative to control plant cells not exposed to said surfactant containing medium.

41. The method of claim 32 wherein said improving transformation of plant cells comprises an increased percentage of callus pieces developing stably transformed sectors.

42. The method of claim 32 wherein said improving transformation of plant cells comprises an increased number of transformed plants regenerated from transformed tissue.

43. The method of claim 32 wherein said plant cells are derived from a monocot.

44. The method of claim 43 wherein said plant cells are derived from Zea mays, Oryza sativa, Setaria viridis, Sorghum bicolor, Triticum aestivum, or Saccharum sp.

45. The method of claim 32 wherein said plant cells are derived from a dicot.

46. The method of claim 45 wherein said plant cells are derived from Pisum sativum, Lactuca sativa, or Solanum lycopersicum.

47. The method of claim 32 wherein said at least one polynucleotide sequence comprises a polynucleotide sequence that shares at least 80% sequence identity with a sequence selected from the group of sequences consisting of SEQ ID NOs:1 and 15, or that encodes a protein that shares at least 80% sequence identity with a sequence selected from the group of sequences consisting of SEQ ID NOs:2 and 16.

48. The method of claim 32 wherein said at least one polynucleotide sequence comprises a polynucleotide sequence that shares at least 80% sequence identity with a sequence selected from the group of sequences consisting of SEQ ID NOs:3, 5, and 7, or that encodes a protein that shares at least 80% sequence identity with a sequence selected from the group of sequences consisting of SEQ ID NO:4, 6, and 8.

49. The method of claim 32 wherein said introducing at least one polynucleotide sequence comprises biolistic transformation.

50. The method of claim 32 wherein said at least one polynucleotide sequence encodes at least one CRISPR nuclease.

51. The method of claim 50 wherein said pre-conditioning results in improved genome editing relative to control cells not exposed to said surfactant containing medium.

52. A method of transforming a seed with a polynucleotide by
i) exposing the seed comprising a seed coat to a surfactant, and
ii) exposing the seed to a bacterium containing the polynucleotide, or a virus containing the polynucleotide,
wherein the bacterium or virus is capable of transforming the plant cell by infecting the plant cell.

53. The method of claim 52 wherein the seed is a whole seed.

54. The method of claim 52 or 53 wherein the seed coat remains attached to the seed.

55. The method of claim 52 or 53 wherein the seed coat remains substantially intact.

56. The method of any one of claims 52-55 wherein the seed has been subjected to sonication.
